(19) United States
(12) Patent Application Publication
(10) Pub. No.: US 2024/0261273 A1
(43) Pub. Date: Aug. 8, 2024

(54) PRIDOPIDINE AND ANALOGS THEREOF FOR THE TREATMENT OF NEURODEGENERATIVE EYE DISEASE

(71) Applicant: PRILENIA NEUROTHERAPEUTICS LTD., Yakum (IL)

(72) Inventors: Michael HAYDEN, Yakum (IL); Michal GEVA, Even-Yehuda (IL)

(73) Assignee: PRILENIA NEUROTHERAPEUTICS LTD., Yakum (IL)

(21) Appl. No.: 18/630,030

(22) Filed: Apr. 9, 2024

Related U.S. Application Data
(63) Continuation-in-part of application No. PCT/IL2022/051082, filed on Oct. 11, 2022.
Application No. PCT/IL2022/051082 is a continuation-in-part of application No. 17/498,075, filed on Oct. 11, 2021.
Application No. 17/498,075 is a continuation-in-part of application No. 17/513,239, filed on Oct. 28, 2021.

(51) Int. Cl. A61K 31/451; A61P 25/28; A61P 27/06

(52) U.S. Cl. CPC A61K 31/451; A61P 25/28; A61P 27/06

(57) ABSTRACT

This application provides a composition comprising pridopidine or pharmaceutically acceptable salt thereof and at least one of compounds 1-8 (described herein) or pharmaceutically acceptable salt thereof for use in the treatment of neurodegenerative eye disease, symptoms thereof, and additional neurodegenerative disorders.

### CROSS-REFERENCE TO RELATED APPLICATIONS

This application is a Continuation in Part Application of PCT International Application No. PCT/IL2022/051082, International Filing Date Oct. 11, 2022; which is a Continuation in Part of United-States application Ser. No. 17/498,075, filed Oct. 11, 2021 and is a Continuation in Part of United-States application Ser. No. 17/513,239, filed Oct. 28, 2021; the entire contents of which are hereby incorporated by reference herein.

### FIELD OF THE INVENTION

This application provides a composition comprising pridopidine or pharmaceutically acceptable salt thereof and at least one of compounds 1-8 (described herein) or pharmaceutically acceptable salt thereof for use in the treatment of neurodegenerative eye disease, symptoms thereof, and additional neurodegenerative disorders.

### BACKGROUND

### Pridopidine

Pridopidine (formerly ACR16, Huntexil®) is a highly potent and highly selective sigma-1 receptor (SiR) agonist in clinical development for neurodegenerative diseases including Huntington disease (HD) and amyotrophic lateral sclerosis (ALS). The chemical name of pridopidine is 4-(3-(Methylsulfonyl) phenyl)-1-propylpiperidine, and its Chemical Registry Number is CAS 346688-38-8 (CSID:7971505, 2016). The Chemical Registry number of pridopidine hydrochloride is 882737-42-0 (CSID:25948790 2016). Processes of synthesis of pridopidine and a pharmaceutically acceptable salt thereof are disclosed in U.S. Pat. No. 7,923,459 and PCT Application Publication No. WO 2017/015609. U.S. Pat. No. 6,903,120 claims pridopidine for the treatment of Parkinson's disease, dyskinesias, dystonias, Tourette's disease, iatrogenic and non-iatrogenic psychoses and hallucinoses, mood and anxiety disorders, sleep disorder, autism spectrum disorder, ADHD, Huntington's disease, age-related cognitive impairment, and disorders related to alcohol abuse and narcotic substance abuse.

### Neurodegenerative Eye Disease

Neurodegeneration of ocular tissues, including the retina and optic nerve leads to vision loss, and blindness when left untreated. Glaucoma is a group of ocular diseases characterized by progressive damage to the eye at least partly due to elevated intraocular pressure (IOP) (Merck Manual of Diagnosis and Therapy (1999)). Additionally, glaucoma is characterized by retinal ganglion cell (RGC) death, axon loss and an excavated appearance of the optic nerve head (Alward 1998). The classification of glaucoma includes several subtypes including for example, primary angle-closure glaucoma, secondary open-angle glaucoma, steroid-induced glaucoma, traumatic glaucoma, pigmentary dispersion syndrome, pseudoexfoliation syndrome, secondary angle-closure glaucoma, neovascular glaucoma, uveitis, and glaucoma and other eye pathologies. Other neurodegenerative diseases of the eye include different forms of macular degeneration, retinitis pigmentosa and all types of optic neuropathy.

Glaucoma can be diagnosed before vision loss occurs by visual field testing and by ophthalmoscopic examination of the optic nerve to detect “cupping.” The mean IOP in normal adults is 15 to 16 mm Hg; the normal range is 10 to 21 mm Hg. One form of management of glaucoma is based on lowering the IOP using topically applied medications (Coleman 1999).

However, despite the availability of treatments for lowering IOP, many patients will continue to lose their vision due to RGC death and degeneration of the optic nerve (Mysona et al. 2017). As a result, therapies aimed at the preservation of RGCs, their axons and dendrites are the focus of many years of research.

Glaucomatous optic neuropathy appears to result from specific pathophysiological changes and subsequent death of RGCs and their axons. The process of RGC death is thought to be biphasic: a primary injury responsible for initiation of damage followed by a slower, secondary degeneration attributable to the hostile environment surrounding the degenerating cells (Kipnis et al. 2000).

The molecular mechanism triggering RGC death has not been identified. Deprivation of neurotrophic factors, ischemia, chronic elevation of glutamate or amyloid beta oligomers and impaired nitric oxide metabolism are suspected to be possible mechanisms (Farkas et al. 2001). In addition, it is possible that the mechanisms leading to RGC death share common features with other types of neuronal injury, such as elevated reactive oxygen species, disrupted Ca2+ homeostasis, ER stress or induction of transcriptionally regulated cell death (Weinreb et al. 1999).

Macular degeneration is a leading cause of progressive vision loss, which affects central vision. Macular degeneration is more likely to occur in older people, hence the name age-related macular degeneration (AMD). AMD is characterized by pathological alterations in the inner retina layers of the macula and adjacent vasculature. There are two types of macular degeneration: dry and wet. Dry (atrophic) macular degeneration is the more prevalent type, affecting ˜90% of people with AMD (Maurya et al., Oxidative stress in retinal pigment epithelium degeneration: from pathogenesis to therapeutic targets in dry age-related macular degeneration. Neural Regeneration Research 18(10): p 2173-2181, October 2023). Yellow protein deposits called drusen accumulate under the macula, causing it to dry and thin. The late, advanced stage of dry AMD is known as geographic atrophy (GA). GA is characterized by the degeneration of retinal pigment epithelial (RPE) cells and photoreceptor death, which leads to atrophic lesions in the outer retina (Nadeem et al., Advancements in the treatment of geographic atrophy: focus on pegcetacoplan in age-related macular degeneration. Ann Med Surg (Lond). 2023 Nov. 1; 85(12):6067-6077).

Wet (exudative) macular degeneration occurs when abnormal blood vessels develop under the retina and macula, leaking blood and fluid that form a bulge in the macula, causing impaired vision.

The neuroprotective effects of pridopidine via S1R activation have been demonstrated in several in vitro and in vivo models of neurodegenerative diseases including HD, Alzheimer's disease (AD), Amyotrophic Lateral Sclorosis (ALS) and Parkinson's disease (PD) (Eddings et al. 2019; Ryskamp et al. 2019; Ionescu et al. 2019; Francardo et al. 2019).

The effects of pridopidine and/or analogs thereof, on neurodegenerative eye diseases, in particular glaucoma, have not been reported previously.

### SUMMARY OF THE INVENTION

The subject invention provides a method of treating, reducing, or inhibiting a neurodegenerative eye disease or a symptom thereof in a subject, comprising administering to the subject a composition comprising pridopidine or pharmaceutically acceptable salt thereof and at least one of compounds 1-8 or pharmaceutically acceptable salt thereof; wherein compounds 1-8 are represented by the following structures:

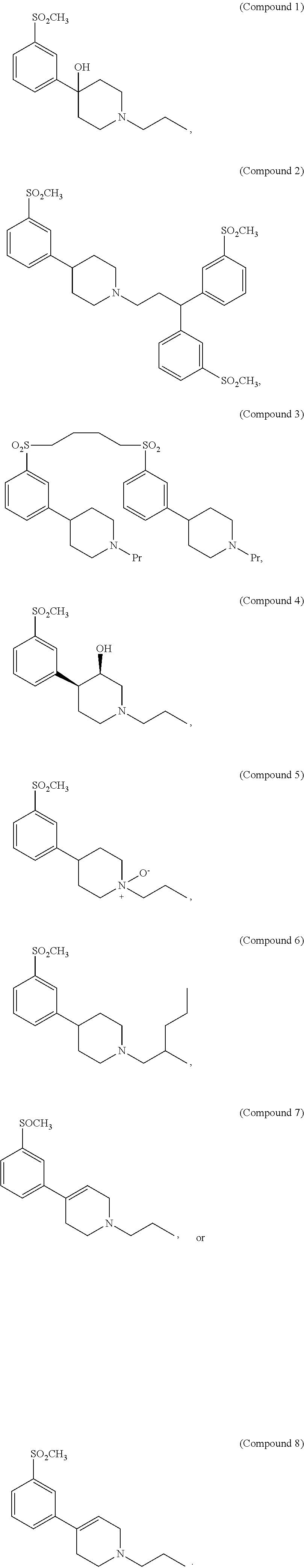

In some embodiments, the composition comprises pridopidine or pharmaceutically acceptable salt thereof and Compound 1 or pharmaceutically acceptable salt thereof. In some embodiments, the composition comprises pridopidine or pharmaceutically acceptable salt thereof and Compound 4 or pharmaceutically acceptable salt thereof. In some embodiments, the composition comprises pridopidine or pharmaceutically acceptable salt thereof and Compound 1 and Compound 4 or pharmaceutically acceptable salt thereof.

In some embodiments, the composition is administered orally. In some embodiments, the composition is administered topically. In some embodiments, the composition is administered intraocularly. In some embodiments, the composition is administered intravitreally. In some embodiments, the composition is administered periodically. In some embodiments, the composition is administered ocularly. In some embodiments, the composition is administered by an eye drop application to the conjunctiva. In some embodiments, the composition is administered in a daily dose comprising an amount of pridopidine between 22.5 mg/day-315 mg/day. In some embodiments, the composition is administered once a day. In some embodiments, the composition is administered twice a day. In some embodiments, the composition is administered three times a day.

In other embodiments, the neurodegenerative eye disease is glaucoma. In other embodiments, the glaucoma is open-angle glaucoma, primary open-angle-glaucoma, angle-closure glaucoma, pigmentary glaucoma, pseudoexfoliative glaucoma, neovascular glaucoma, steroid-induced glaucoma, normal-tension glaucoma, pressure-independent glaucoma or any combination thereof. In other embodiments, the neurodegenerative eye disease is Age-related Macular Degeneration (AMD). In some embodiments, the Age-related Macular Degeneration is Wet Age-related Macular Degeneration (“Wet AMD”). In some embodiments, the Age-related Macular Degeneration is Dry Age-related Macular Degeneration (“Dry AMD”). In other embodiments, the neurodegenerative eye disease is geographic atrophy (GA). In other embodiments, the neurodegenerative eye disease is optic neuropathy. In some embodiments, optic neuropathy is Leber hereditary optic neuropathy. In other embodiments, the neurodegenerative eye disease is Microphthalmia, syndromic 12 (MCOPS12). In other embodiments, the neurodegenerative eye disease is retinitis pigmentosa.

In other embodiments, the symptom is retinal ganglion cell damage. In other embodiments, the symptom is retinal ganglion cell loss. In other embodiments, the symptom is an optic nerve axon loss or damage. In other embodiments, the symptom is macular degeneration. In other embodiments, the symptom is a retinal ganglion cell (RGC) loss or damage/death. In other embodiments, the symptom is a retinal pigment epithelium cell (RPE) loss or death. In other embodiments, the symptom is an optic nerve head astrocyte (ONHA) loss or damage.

In other embodiments, the composition is effective in reducing or preventing retinal ganglion cell (RGC) loss or damage in a subject. In other embodiments, the composition is effective in improving retinal ganglion cell (RGC) viability in the subject. In other embodiments, the composition is effective in reducing or preventing optic nerve axon loss or damage in a subject. In other embodiments, the composition is effective in reducing or preventing optic nerve head astrocytes (ONHAs) loss or damage in the subject. In other embodiments, the composition is effective in reducing oxidative stress in ONHAs. In other embodiments, the composition is effective in improving optic nerve head astrocytes (ONHAs) viability in the subject. In other embodiments, the composition is effective in increasing cell viability in the subject. In other embodiments, the composition is effective in reducing or preventing retinal pigment epithelium cell (RPE) loss or death in a subject. In other embodiments, the composition is effective in protecting an optic nerve axon from degeneration in the subject. In other embodiments, the axon degeneration is induced by elevated intraocular pressure.

In other embodiments, the cell viability is increased by at least 3%, by at least 5%, by at least 10%, by at least 20%, or by at least 25%. In other embodiments, the optic nerve axon loss is reduced by at least 3%, by at least 5%, by at least 10%, by at least 20%, by at least 30%, by at least 40% or by at least 50%. In other embodiments, the optic nerve axon loss is reduced by more than 50%, more than 60%, more than 70%, or more than 80%. In other embodiments, the retinal ganglion cell loss is reduced by at least 10%, by at least 20%, by at least 30%, by at least 40% or by at least 50%. In other embodiments, the retinal ganglion cell loss is reduced by more than 50%, more than 60%, more than 70%, or more than 80%. %. In other embodiments, the optic nerve head astrocyte (ONHA) loss is reduced by at least 3%, by at least 5%, by at least 10%, by at least 20%, by at least 25%, by at least 30%, by at least 35%, by at least 40% or by at least 50%. In other embodiments, the composition is effective in improving retinal ganglion cell viability in a subject. In other embodiments, the composition is effective in improving optic nerve head astrocyte (ONHA) viability in a subject. In other embodiments, the composition is effective in protecting an optic nerve axon from degeneration in the subject. In other embodiments, the axon degeneration is induced by elevated intraocular pressure.

The subject invention also provides a method of preventing or reducing retinal pigment epithelium cell damage or loss in a subject, comprising administering to the subject a pharmaceutical composition comprising pridopidine or a pharmaceutically acceptable salt thereof and at least one of compounds 1-8 or pharmaceutically acceptable salt thereof:

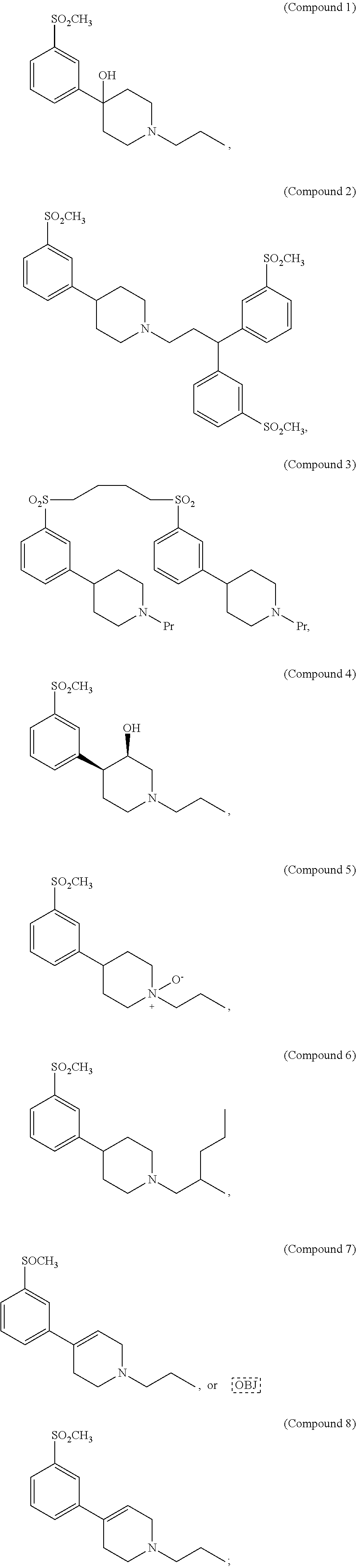

effective to prevent or reduce retinal ganglion cell damage or loss in the subject.

The subject invention provides a method of treating neurodegenerative eye disease in a subject comprising administering to the subject a pharmaceutical composition comprising pridopidine or a pharmaceutically acceptable salt thereof and at least one of compounds 1-8 or pharmaceutically acceptable salt thereof:

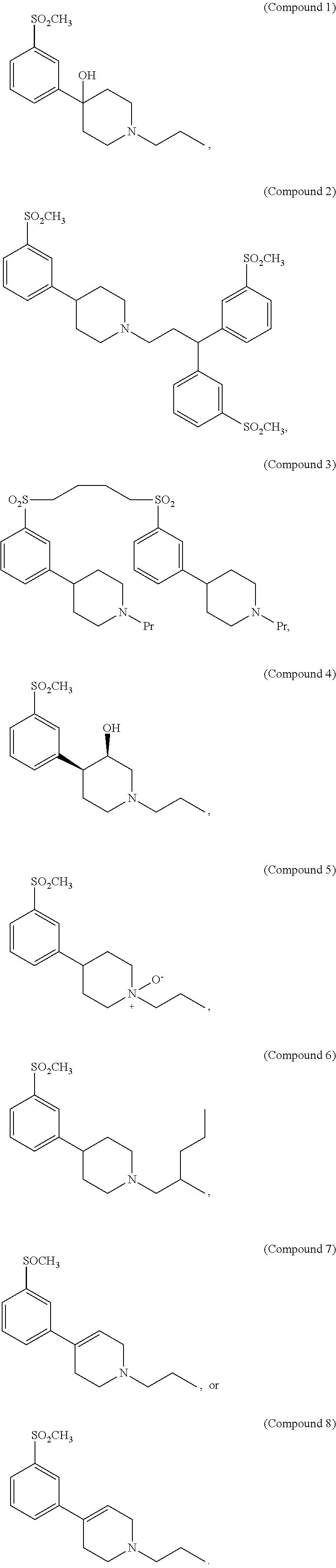

effective to provide neuroprotection to a retinal ganglion cell in the subject.

The subject invention also provides a pharmaceutical composition comprising an amount of pridopidine or a pharmaceutically acceptable salt thereof and at least one compound of formula 1-8 or pharmaceutically acceptable salt thereof, for treating a subject afflicted with a neurodegenerative eye disease.

The subject invention also provides a pharmaceutical composition comprising pridopidine or a pharmaceutically acceptable salt thereof and at least one of compounds 1-8 or pharmaceutically acceptable salt thereof, for use in a combination therapy together with a second pharmaceutical composition comprising a second agent for the treatment of a neurodegenerative eye disease.

The subject invention also provides a pharmaceutical composition comprising an amount of pridopidine or a pharmaceutically acceptable salt thereof and at least one of compounds 1-8 or pharmaceutically acceptable salt thereof, for use in treating a subject afflicted with a neurodegenerative eye disease as an add-on therapy or in combination with a second agent for the treatment of a neurodegenerative eye disease.

The subject invention also provides a pharmaceutical composition in a unit dosage form, useful in treating a subject afflicted with a neurodegenerative eye disease, which comprises an amount of pridopidine or pharmaceutically acceptable salt thereof and at least one of compounds 1-8 or pharmaceutically acceptable salt thereof, wherein the amount of said pridopidine in said composition is effective, upon administration to said subject of one or more of said unit dosage forms of said composition, to treat the subject.

### BRIEF DESCRIPTION OF THE FIGURES

FIGS. 1A-1B: Synergistic effect of pridopidine and Compound 4 on BDNF Release from B104 cells. B104 neuroblastoma cells were incubated for 5 days with test compounds, and BDNF levels were assessed using in-situ ELISA. FIG. 1A: Pridopidine at a concentration of 0.001 μM and Compound 4 at a concentration of 0.001 μM. Pridopidine alone increased BDNF secretion by 13.5%. Compound 4 alone reduced BDNF secretion by −1.5%. Pridopidine and compound 4 together increased BDNF secretion by 59.1%, an effect which is greater than the added effect of both compounds administered on their own. FIG. 1B: Pridopidine at a concentration of 0.005 μM and Compound 4 at a concentration of 0.001 μM. Pridopidine alone increased BDNF secretion by 26.0%. Compound 4 alone reduced BDNF secretion by −1.5%. Pridopidine and compound 4 together increased BDNF secretion by 80.7%, an effect which is greater than the added effect of both compounds administered on their own.

FIG. 2: Synergistic effect of pridopidine and Compound 1 on BDNF Release from B104 cells. B104 neuroblastoma cells were incubated for 5 days with test compounds, and BDNF levels were assessed using in-situ ELISA. Pridopidine at a concentration of 0.01 μM alone increased BDNF secretion by 3.4%. Compound 1 at a concentration of 1 μM alone increased BDNF secretion by 12.5%. Pridopidine and compound 1 together increased BDNF secretion by 53.1%, an effect which is greater than the added effect of both compounds administered on their own.

FIGS. 3-5: Combination effects of pridopidine and compound 1 and compound 4 on oxidative stress levels and survival of optic nerve head astrocytes. Astrocytes provide essential support to neurons in the central nervous system, and their dysfunction is associated with neurodegenerative processes. Healthy optic nerve head astrocytes (ONHA) have been shown to protect RGCs, while glaucomatous ONHAs have been shown to induce neurodegenerative phenotypes. Like RGCs, ONHAs are responsive to cases of IOP elevation and oxidative stress. Thus, ONHAs are a valid model for preclinical assessment of the effects of ocular treatments.

FIG. 3: Combination of pridopidine and compound 1 increases cell viability in a synergistic manner. ONHAs were incubated for 16 hours with vehicle, pridopidine (0.05 μM), compound 1 (0.01 μM) or a combination of the two, then oxidative stress was induced with tert-butyl hydroperoxide (tBHP, 40 μM) for 6 hours. Cell viability was assessed using the MTT colorimetric assay. Neither pridopidine nor compound 1 had any effect on cell viability when administered alone. Surprisingly, in combination, pridopidine and compound 1 increase cell viability by 27%. Thus, the two compounds provide a synergistic effect.

FIG. 4: Combination of pridopidine and compound 4 increases cell viability in a synergistic manner. ONHAs were incubated for 16 hours with vehicle, pridopidine (0.05 μM), compound 4 (0.1 μM) or a combination of the two, then oxidative stress was induced with tert-butyl hydroperoxide (tBHP, 40 μM) for 6 hours. Cell viability was assessed using the MTT colorimetric assay. Neither pridopidine nor compound 4 had any effect on cell viability when administered alone. Surprisingly, in combination, pridopidine and compound 4 increase cell viability by 26%. Thus, the two compounds provide a synergistic effect.

FIG. 5: Additive effect of pridopidine and compound 1 reducing oxidative stress in ONHAs. ONHAs were incubated for 16 hours with vehicle, pridopidine (0.05 μM), compound 1 (0.05 μM) or a combination of the two, then oxidative stress was induced with tert-butyl hydroperoxide (tBHP, 80 μM) for 6 hours. Oxidative stress was assessed by measuring DCF fluorescence, which is indicative of toxic reactive oxygen species (ROS) levels. tBHP induces a 5-fold increase DCF fluorescence levels (p<0.0001), indicating an increase in oxidative stress. Pridopidine alone reduces oxidative stress by 30% (p<0.001) and compound 1 by 5%. The combination of pridopidine and compound 1 demonstrates enhanced effect with reduction of 35% in oxidative stress, indicating an additive effect of the two.

### DETAILED DESCRIPTION OF THE INVENTION

This invention provides a method of treating, slowing the progression, lessening the decline, delaying onset of symptoms, or slowing the progression of symptoms of a neurodegenerative or neurodevelopmental disease or disorder in a subject; wherein the method comprises administering a composition comprising pridopidine or a pharmaceutically acceptable salt thereof and at least one of Compounds 1-8 or pharmaceutically acceptable salt thereof:

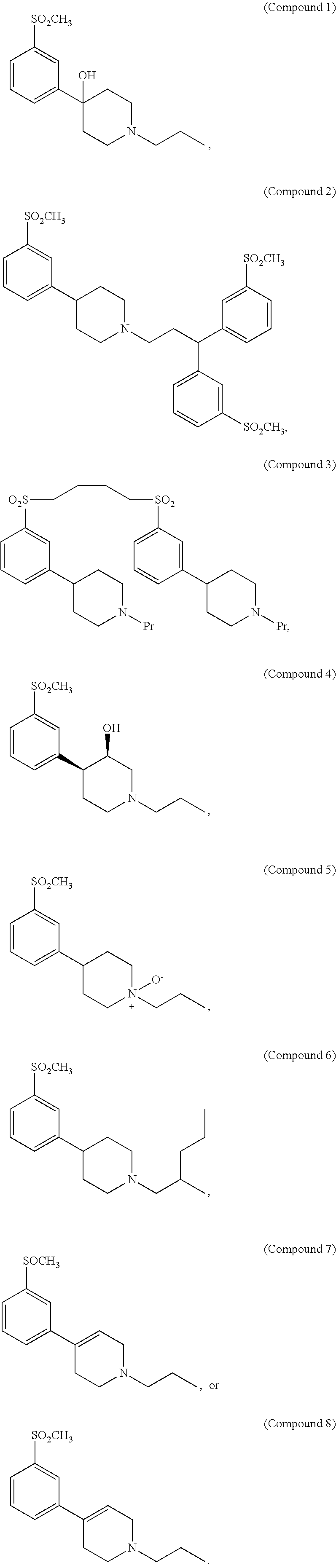

In some embodiments, the neurodegenerative disease or disorder is selected from the group consisting from Huntington Disease, prodromal/premanifest Huntington disease, Amyotrophic Lateral Sclerosis (ALS), Parkinson's Disease, Parkinson's Disease associated with glucocerebrosidase (GBA) deficiency, dystonia, cognitive disorder, dyskinesia, mild cognitive impairment (MCI), Alzheimer's Disease, age related memory loss, depression and anxiety, bacterial infections-induced depression, neurodegenerative eye disease, optic neuropathies including glaucoma, age-related macular degeneration (AMD), Leber's Hereditary Optic Neuropathy (LHON), and retinitis pigmentosa, Microphthalmia, syndromic 12 (MCOPS12), mitochondrial diseases or dysfunctions (i.e. Lysosomal storage disease (LSD), leukodystrophies, vanishing white matter (VWM) disease), Wolfram disease and viral infection (i.e. COVID-19); and wherein the neurodevelopmental disease or disorder is Rett syndrome or Fragile X Syndrome.

In some embodiments, the neurodegenerative disease or disorder is a neurodegenerative eye disease. In some embodiments, the neurodegenerative eye disease is selected from the group consisting of glaucoma, Age-related Macular Degeneration (AMD), geographic atrophy (GA), optic neuropathy, Microphthalmia, syndromic 12 (MCOPS12), and retinitis pigmentosa. In another embodiment, the neurodegenerative eye disease refers to any disease affecting retinal ganglion cells, photoreceptors, other retinal neurons, and corneal nerves. In one embodiment, the neurodegenerative eye disease is glaucoma. In another embodiment, the neurodegenerative eye disease is Wet Age-related Macular Degeneration (“Wet AMD”), Dry Age-related Macular Degeneration (“Dry AMD”) or geographic atrophy (GA). In a further embodiment, the neurodegenerative eye disease is Leber hereditary optic neuropathy (LHON).

In some embodiments, this invention further provides a method of treating, reducing, or inhibiting a neurodegenerative eye disease or a symptom thereof in a subject comprising administering to the subject a composition comprising pridopidine or pharmaceutically acceptable salt thereof and at least one of compounds 1-8 as described hereinabove, or pharmaceutically acceptable salt thereof.

In some embodiments, the symptom is retinal ganglion cell (RGC) damage. In other embodiments, the symptom is retinal ganglion cell (RGC) loss. In other embodiments, the symptom is an optic nerve axon loss or damage. In other embodiments, the symptom is macular degeneration. In other embodiments, the symptom is a retinal ganglion cell (RGC) loss or damage/death. In other embodiments, the symptom is a retinal pigment epithelium cell (RPE) loss or death. In other embodiments, the symptom is photoreceptor loss or death. In other embodiments, the composition is effective to reduce or prevent optic nerve axon loss or damage in a subject. In other embodiments, the composition is effective to reduce or prevent a retinal ganglion cell (RGC) loss or death in a subject. In other embodiments, the composition is effective to reduce or prevent a retinal pigment epithelium (RPE) cell loss or death in a subject. In other embodiments, the composition is effective to reduce or prevent an optic nerve head astrocyte (ONHA) loss or damage. In other embodiments, the composition is effective to reduce or prevent photoreceptor loss or death in a subject.

In some embodiments, the composition comprising pridopidine or a pharmaceutically acceptable salt thereof and at least one of Compounds 1-8 or pharmaceutically acceptable salt thereof for use in the methods of this invention. In some embodiments, the composition comprises a weight ratio between the pridopidine and at least one of compounds 1-8 in the range of 1:0.0001 to 1:0.1. In other embodiments, the weight ratio between the pridopidine or a pharmaceutically acceptable salt thereof and at least one of compounds 1-8 or a pharmaceutically acceptable salt thereof is in the range of 1:0.0005 to 1:0.005. In other embodiments, the weight ratio between the pridopidine or a pharmaceutically acceptable salt thereof and at least one of compounds 1-8 or a pharmaceutically acceptable salt thereof is in the range of 1:0.0005 to 1:0.0035. In other embodiments, the weight ratio between the pridopidine or a pharmaceutically acceptable salt thereof and at least one of compounds 1-8 or a pharmaceutically acceptable salt thereof is in the range of 1:0.005 to 1:0.1. In other embodiments, the weight ratio between the pridopidine or a pharmaceutically acceptable salt thereof and at least one of compounds 1-8 or a pharmaceutically acceptable salt thereof is in the range of 1:0.001 to 1:0.1. In other embodiments, the weight ratio between the pridopidine or a pharmaceutically acceptable salt thereof and at least one of compounds 1-8 or a pharmaceutically acceptable salt thereof is in the range of 1:0.001 to 1:0.005. In other embodiments, the weight ratio between the pridopidine or a pharmaceutically acceptable salt thereof and at least one of compounds 1-8 or a pharmaceutically acceptable salt thereof is in the range of 1:10 to 10:1. In other embodiments, the weight ratio between the pridopidine or a pharmaceutically acceptable salt thereof and at least one of compounds 1-8 or a pharmaceutically acceptable salt thereof is in the range of 1:5 to 5:1. In other embodiments, the weight ratio between the pridopidine or a pharmaceutically acceptable salt thereof and at least one of compounds 1-8 or a pharmaceutically acceptable salt thereof is in the range of 1:100 to 5:1.

In other embodiments, the composition comprising pridopidine or a pharmaceutically acceptable salt thereof and at least one of Compounds 1-8 or pharmaceutically acceptable salt thereof for use in the methods of this invention is administered in a daily dose comprising between 0.5-315 mg pridopidine or a pharmaceutically acceptable salt thereof. In another embodiment, composition is an oral dosage form administered in a daily dose comprising 0.5-315 mg pridopidine or a pharmaceutically acceptable salt thereof. In another embodiment, the oral dosage unit form is administered in a daily dose comprising 1-10 mg pridopidine or a pharmaceutically acceptable salt thereof. In another embodiment, the oral dosage unit form is administered in a daily dose comprising 10-22.5 mg pridopidine or a pharmaceutically acceptable salt thereof. In another embodiment, the oral dosage unit form is administered in a daily dose comprising 22.5-315 mg pridopidine or a pharmaceutically acceptable salt thereof. In another embodiment, the oral dosage unit form is administered in a daily dose comprising 10-315 mg pridopidine or a pharmaceutically acceptable salt thereof. In another embodiment, the oral dosage unit form is administered in a daily dose comprising 0.5-50 mg pridopidine or a pharmaceutically acceptable salt thereof. In another embodiment, the oral dosage unit form the oral dosage unit form is administered in a daily dose comprising 45-250 mg pridopidine or a pharmaceutically acceptable salt thereof. In another embodiment, the oral dosage unit form is administered in a daily dose comprising 45-135 mg pridopidine or a pharmaceutically acceptable salt thereof. In another embodiment, the oral dosage unit form is administered in a daily dose comprising 90-315 mg pridopidine or a pharmaceutically acceptable salt thereof.

In other embodiments, the method of treating, slowing the progression, lessening the decline, delaying onset of symptoms or slowing the progression of symptoms of neurodegenerative or neurodevelopmental disease or disorder in a subject comprises administering a composition comprising pridopidine or a pharmaceutically acceptable salt thereof and Compound 1 or pharmaceutically acceptable salt thereof. In other embodiment, the composition comprises pridopidine or a pharmaceutically acceptable salt thereof and Compound 2 or pharmaceutically acceptable salt thereof. In other embodiment, the composition comprises pridopidine or a pharmaceutically acceptable salt thereof and Compound 3 or pharmaceutically acceptable salt thereof. In other embodiment, the composition comprises pridopidine or a pharmaceutically acceptable salt thereof and Compound 4 or pharmaceutically acceptable salt thereof. In other embodiment, the composition comprises pridopidine or a pharmaceutically acceptable salt thereof and Compound 5 or pharmaceutically acceptable salt thereof. In other embodiment, the composition comprises pridopidine or a pharmaceutically acceptable salt thereof and Compound 6 or pharmaceutically acceptable salt thereof. In other embodiment, the composition comprises pridopidine or a pharmaceutically acceptable salt thereof and Compound 7 or pharmaceutically acceptable salt thereof. In other embodiment, the composition comprises pridopidine or a pharmaceutically acceptable salt thereof and Compound 8 or pharmaceutically acceptable salt thereof. In other embodiment, the composition comprises pridopidine or a pharmaceutically acceptable salt thereof, Compound 1 or pharmaceutically acceptable salt thereof and Compound 4 or pharmaceutically acceptable salt thereof. In other embodiment, the composition comprises a pridopidine base. In other embodiment, the composition comprises a pridopidine salt.

In other embodiments, the method of treating, reducing or inhibiting a neurodegenerative eye disease or a symptom thereof in a subject comprises administering a composition comprising pridopidine or a pharmaceutically acceptable salt thereof and Compound 1 or pharmaceutically acceptable salt thereof. In other embodiment, the composition comprises pridopidine or a pharmaceutically acceptable salt thereof and Compound 2 or pharmaceutically acceptable salt thereof. In other embodiment, the composition comprises pridopidine or a pharmaceutically acceptable salt thereof and Compound 3 or pharmaceutically acceptable salt thereof. In other embodiment, the composition comprises pridopidine or a pharmaceutically acceptable salt thereof and Compound 4 or pharmaceutically acceptable salt thereof. In other embodiment, the composition comprises pridopidine or a pharmaceutically acceptable salt thereof and Compound 5 or pharmaceutically acceptable salt thereof. In other embodiment, the composition comprises pridopidine or a pharmaceutically acceptable salt thereof and Compound 6 or pharmaceutically acceptable salt thereof. In other embodiment, the composition comprises pridopidine or a pharmaceutically acceptable salt thereof and Compound 7 or pharmaceutically acceptable salt thereof. In other embodiment, the composition comprises pridopidine or a pharmaceutically acceptable salt thereof and Compound 8 or pharmaceutically acceptable salt thereof. In other embodiment, the composition comprises pridopidine or a pharmaceutically acceptable salt thereof, Compound 1 or pharmaceutically acceptable salt thereof and Compound 4 or pharmaceutically acceptable salt thereof. In other embodiment, the composition comprises a pridopidine base. In other embodiment, the composition comprises a pridopidine salt.

In some embodiments this invention provides a method of treating, slowing the progression, lessening the decline, delaying onset of symptoms or slowing the progression of symptoms of a neurodegenerative or neurodevelopmental disease or disorder in a subject; wherein the method comprises administering a composition comprising pridopidine or a pharmaceutically acceptable salt thereof and at least one of Compound 1 or pharmaceutically acceptable salt thereof, Compound 4 or pharmaceutically acceptable salt thereof; or combination thereof; wherein the neurodegenerative disease or disorder is selected from the group consisting from Huntington Disease, prodromal/premanifest Huntington disease, Amyotrophic Lateral Sclerosis (ALS), Parkinson's Disease, Parkinson's Disease associated with glucocerebrosidase (GBA) deficiency, dystonia, cognitive disorder, dyskinesia, mild cognitive impairment (MCI), Alzheimer's Disease, age related memory loss, depression and anxiety, bacterial infections-induced depression, neurodegenerative eye disease, optic neuropathies including glaucoma, age-related macular degeneration (AMD), Leber's Hereditary Optic Neuropathy (LHON), Microphthalmia, syndromic 12 (MCOPS12), and retinitis pigmentosa, mitochondrial diseases or dysfunctions (i.e. Lysosomal storage disease (LSD), leukodystrophies, vanishing white matter (VWM) disease), Wolfram Disease, and viral infection (i.e. COVID-19); and wherein the neurodevelopmental disease or disorder is Rett syndrome or Fragile X Syndrome. In other embodiment, the composition comprises a pridopidine base. In other embodiment, the composition comprises a pridopidine salt.

In some embodiments this invention provides a method of treating, reducing or inhibiting a neurodegenerative eye disease or a symptom thereof in a subject; wherein the method comprises administering a composition comprising pridopidine or a pharmaceutically acceptable salt thereof and at least one of Compound 1 or pharmaceutically acceptable salt thereof, Compound 4 or pharmaceutically acceptable salt thereof; or combination thereof.

In some embodiments this invention provides a method of treating, slowing the progression, lessening the decline, delaying onset of symptoms or slowing the progression of symptoms of a neurodegenerative or neurodevelopmental disease or disorder in a subject; wherein the method comprises administering a composition comprising pridopidine or a pharmaceutically acceptable salt thereof and Compound 1 or pharmaceutically acceptable salt thereof; wherein the neurodegenerative disease or disorder is selected from the group consisting from Huntington Disease, prodromal/premanifest Huntington disease, Amyotrophic Lateral Sclerosis (ALS), Parkinson's Disease, Parkinson's Disease associated with glucocerebrosidase (GBA) deficiency, dystonia, cognitive disorder, dyskinesia, mild cognitive impairment (MCI), Alzheimer's Disease, age related memory loss, depression and anxiety, neurodegenerative eye disease, bacterial infections-induced depression, optic neuropathies including glaucoma, age-related macular degeneration (AMD), Leber's Hereditary Optic Neuropathy (LHON) and retinitis pigmentosa, Microphthalmia, syndromic 12 (MCOPS12), mitochondrial diseases or dysfunctions (i.e. Lysosomal storage disease (LSD), leukodystrophies, vanishing white matter (VWM) disease), Wolfram Disease and viral infection (i.e. COVID-19); and wherein the neurodevelopmental disease or disorder is Rett syndrome or Fragile X Syndrome. In other embodiment, the composition comprises a pridopidine base. In other embodiment, the composition comprises a pridopidine salt.

In some embodiments this invention provides a method of treating, reducing or inhibiting a neurodegenerative eye disease or a symptom thereof in a subject; wherein the method comprises administering a composition comprising pridopidine or a pharmaceutically acceptable salt thereof and Compound 1 or pharmaceutically acceptable salt thereof.

In some embodiments this invention provides a method of treating, slowing the progression, lessening the decline, delaying onset of symptoms or slowing the progression of symptoms of a neurodegenerative or neurodevelopmental disease or disorder in a subject; wherein the method comprises administering a composition comprising pridopidine, Compound 1 and Compound 4 or pharmaceutically acceptable salt thereof; wherein the neurodegenerative disease or disorder is selected from the group consisting from Huntington Disease, prodromal/premanifest Huntington disease, Amyotrophic Lateral Sclerosis (ALS), Parkinson's Disease, Parkinson's Disease associated with glucocerebrosidase (GBA) deficiency, dystonia, cognitive disorder, dyskinesia, mild cognitive impairment (MCI), Alzheimer's Disease, age related memory loss, depression and anxiety, bacterial infections-induced depression, neurodegenerative eye disease, optic neuropathies including glaucoma, age-related macular degeneration (AMD), Leber's Hereditary Optic Neuropathy (LHON) and retinitis pigmentosa, Microphthalmia, syndromic 12 (MCOPS12), mitochondrial diseases or dysfunctions (i.e. Lysosomal storage disease (LSD), leukodystrophies, vanishing white matter (VWM) disease), Wolfram Disease and viral infection (i.e. COVID-19); and wherein the neurodevelopmental disease or disorder is Rett syndrome or Fragile X Syndrome. In other embodiment, the composition comprises a pridopidine base. In other embodiment, the composition comprises a pridopidine salt.

In some embodiments this invention provides a method of treating, reducing or inhibiting a neurodegenerative eye disease or a symptom thereof in a subject; wherein the method comprises administering a composition comprising pridopidine or a pharmaceutically acceptable salt thereof and Compound 1 and Compound 4 or pharmaceutically acceptable salt thereof.

In some embodiments this invention provides a method of treating, slowing the progression, lessening the decline, delaying onset of symptoms or slowing the progression of symptoms of a neurodegenerative or neurodevelopmental disease or disorder in a subject; wherein the method comprises administering a composition comprising pridopidine, Compound 4 or pharmaceutically acceptable salt thereof; wherein the neurodegenerative disease or disorder is selected from the group consisting from Huntington Disease, prodromal/premanifest Huntington disease, Amyotrophic Lateral Sclerosis (ALS), Parkinson's Disease, Parkinson's Disease associated with glucocerebrosidase (GBA) deficiency, dystonia, cognitive disorder, dyskinesia, mild cognitive impairment (MCI), Alzheimer's Disease, age related memory loss, depression and anxiety, bacterial infections-induced depression, neurodegenerative eye disease, optic neuropathies including glaucoma, age-related macular degeneration (AMD), Leber's Hereditary Optic Neuropathy (LHON) and retinitis pigmentosa, Microphthalmia, syndromic 12 (MCOPS12), mitochondrial diseases or dysfunctions (i.e. Lysosomal storage disease (LSD), leukodystrophies, vanishing white matter (VWM) disease), Wolfram Disease and viral infection (i.e. COVID-19); and wherein the neurodevelopmental disease or disorder is Rett syndrome or Fragile X Syndrome. In other embodiment, the composition comprises a pridopidine base. In other embodiment, the composition comprises a pridopidine salt.

In some embodiments this invention provides a method of treating, reducing or inhibiting a neurodegenerative eye disease or a symptom thereof in a subject; wherein the method comprises administering a composition comprising pridopidine or a pharmaceutically acceptable salt thereof and Compound 4 or pharmaceutically acceptable salt thereof.

In another embodiment the method of this invention is directed to delaying the onset of symptoms in prodromal/premanifest Huntington disease individuals which have ≥36 CAG repeats in the huntingtin gene, comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

In other embodiments, the methods of this invention are directed to treating or slowing the progression of symptoms of prodromal/premanifest Huntington disease.

In other embodiments, the methods of this invention provide maintaining, reducing, or lessening the increase of Neurofilament light protein (NfL) in biofluids (i.e. cerebrospinal fluid, blood and plasma). In other embodiments, the method of this invention provides maintaining, reducing, or lessening the increase of Neurofilament light protein (NfL) in biofluids (i.e. cerebrospinal fluid, blood and plasma) in a neurodegenerative disease including Huntington disease, ALS and Parkinson's disease patients.

In other embodiments, the methods of this invention are directed to treating, slowing the progression, lessening the decline, delaying onset of symptoms, or slowing the progression of symptoms of Parkinson's disease comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

In other embodiments, the methods of this invention are directed to treating, slowing the progression, lessening the decline, delaying onset of symptoms or slowing the progression of symptoms of Parkinson's disease associated with glucocerebrosidase (GBA) deficiency comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In another embodiment, the composition comprises a pridopidine salt.

In other embodiments, the methods of this invention are directed to treating, slowing the progression, lessening the decline, delaying onset of symptoms or slowing the progression of symptoms of Parkinson's disease, a disease associated with parkinsonism, or Parkinson's disease associate with glucocerebrosidase (GBA) deficiency comprising administering a composition comprising pridopidine or a pharmaceutically acceptable salt thereof and at least one of Compounds 1-8 or pharmaceutically acceptable salt thereof. In other embodiments the symptoms comprises a functional decline, cognitive decline. In certain embodiments, the functional decline of the subject is presented as a symptom selected from the group consisting of tremor, bradykinesia, rigidity, postural instability, a decline according to the Unified Parkinson's Disease Rating Scale part II (UPDRS part II), including Activities of Daily living, and a decline according to the Modified Hoehn and Yahr Staging of PD. In certain embodiments, the functional decline of the subject is presented as a decline according to the Unified Parkinson's Disease Rating Scale part II (UPDRS part II), including Activities of Daily living. In certain embodiments, the functional decline of the subject is presented as a decline according to the Modified Hoehn and Yahr Staging of PD.

In certain embodiments, the cognitive decline of the subject is presented as a symptom selected from the group consisting of intellectual impairment, thought disorder, depression, decreased motivation, decreased initiative, impaired speech, increased salvation, impaired swallowing, impaired handwriting, and increased pain sensation.

In other embodiments, the methods of this invention are directed to treating, slowing the progression, lessening the decline, delaying onset of symptoms or slowing the progression of symptoms dystonia comprising administering a composition comprising pridopidine or a pharmaceutically acceptable salt thereof and at least one of Compounds 1-8 or pharmaceutically acceptable salt thereof. In another embodiment, the dystonia is severe dystonia. In other embodiments the symptoms of dystonia comprise involuntary limb movement or muscle contractions, twisted posture of the limbs or trunk, abnormal fixed posture of the limbs or trunk, talipes equinovarus, turning in of the leg, turning in of the arm, tremor of the hand, head, trunk or arms, dragging of the leg, torticollis, writer's cramp, or dystonia of trunk and/or extremities. In another embodiment, the dystonia is a severe dystonia. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 1 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In another embodiment, the composition comprises a pridopidine salt.

“Severe dystonia” may be determined by Burke-Fahn-Marsden Dystonia Rating Scale (BFMDRS) having Rating Scale ≥4 for at least one body part. Burke-Fahn-Marsden Dystonia Rating Scale (BFMDRS) evaluates nine body parts (eyes, mouth, speech, swallowing, neck, trunk, right arm, right leg, left arm, and left leg) by rating the severity factor and provoking factors for each part on a 5-point scale of 0 (no dystonia) to 4 (indicating the presence of dystonia at rest). The dystonia scores of the eyes, mouth and neck are assigned a weighting factor of 0.5, while the other 6 parts are assigned a weighting factor of 1.0. The score of each part is obtained by multiplying the provoking factor by the severity factor and the weighting factor, and then summing the scores of each part. The maximum score possible is 120.

Severe dystonia may also be determined by the Unified Dystonia Rating Scale (UDRS) Rating Scale having Rating Scale ≥4 for at least one body part. UDRS evaluates 14 body parts (eyes and upper face, lower face, jaw and tongue, larynx, neck, trunk, right shoulder/proximal arm, left shoulder/proximal arm, right distal arm/hand, left distal arm/hand, right proximal leg, left proximal leg, right distal leg/foot, and left distal leg/foot) by rating the severity and duration factors for each part. The severity factor for each part is rated using a 5-point scale, ranging from 0 (no dystonia) to 4 (severe dystonia). The duration factor is rating on a 5 point scale ranging from 0 (at rest/action) to 4 (submaximal/maximal). The total score is the sum of each domain (part), with the maximum being 112.

In other embodiments, the methods of this invention are directed to treating, slowing the progression, lessening the decline, delaying onset of symptoms or slowing the progression of symptoms of a cognitive disorder comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In another embodiment, the composition comprises a pridopidine salt.

In certain embodiments the cognitive disorder is mild cognitive impairment. In certain embodiments, the cognitive disorder comprises memory loss. In certain embodiments, the cognitive disorder comprises age related memory loss.

Cognitive disorder refers to impairment of cognitive function which is selected from the group consisting of global cognitive functioning, sustained cognition, memory, language, executive functioning, and attention. In another embodiment, the cognitive function is memory. In an embodiment, memory is short term memory. In another embodiment, memory is long term memory. In another embodiment, memory is working memory. In an embodiment, the subject is afflicted with a cognitive deficit. In another embodiment, the subject is prone to or predisposed to have a cognitive deficit. In an embodiment, the cognitive deficit is a memory deficit. In an embodiment, the memory deficit is a short-term memory deficit. In another embodiment, the memory deficit is memory loss. In an embodiment, memory loss is caused by one or more of age-related changes in memory, mild cognitive impairment, dementia, or depression. In an embodiment, the cognitive deficit is caused by or associated with a disease or disorder. In an embodiment, the disease or disorder is a disease or disorder associated with NMDA receptor. In another embodiment, the disease or disorder is schizophrenia or autism. In another embodiment, the disease or disorder is epilepsy or an anxiety disorder. In another embodiment, the disease or disorder is amyotrophic lateral sclerosis (ALS). In another embodiment, the disease or disorder is frontotemporal dementia (FTD). In another embodiment, the disease or disorder is mild cognitive impairment (MCI). In another embodiment, the disease or disorder is bipolar disorder. In another embodiment, the disease or disorder is Huntington disease. In another embodiment, the disease or disorder is selected from the group consisting of major depressive disorder (MDD), Parkinson's disease, Alzheimer's disease, tardive dyskinesia, depression, sickle cell anemia, stroke, chronic pain syndrome, and addiction. In another embodiment, the disease or disorder is selected from the group consisting of mild cognitive impairment, memory loss, memory deficit, a memory deficit related to brain injury or a post-stroke event, a learning deficiency, and behavioral and cognitive problems associated with brain tumors. In another embodiment, the disease or disorder is selected from the group consisting of dementia, dementia associated with Lewy Bodies, age-related cognitive decline, psychosis, attention deficit disorder (ADHD), bipolar disorder, brain injury, mood and affective disorders, Tourette's syndrome, mental retardation, progressive supranuclear palsy, Creutzfeldt-Jacob disease, corticobasal Degeneration, vascular dementia, and Pick's disease. In another embodiment, the disease or disorder is selected from the group consisting of generalized anxiety disorder (GAD), social anxiety disorder (SAD), tardive dyskinesia, depression, sickle cell anemia, chronic pain syndrome, addiction, nicotine addiction, internet addiction, cocaine addiction, tourette's syndrome, mental retardation, corticobasal degeneration, vascular dementia, Pick's disease, posttraumatic stress disorder (PTSD), obsessive compulsive disorder, panic disorder (PD), trigeminal pain, trigeminal musculoskeletal pain, phantom limb pain, irritable bowel syndrome, blepharospasm, complex regional pain syndrome, chronic low back pain, autism spectrum disorder (ASD), infantile spasm (IS).

In other embodiments, the methods of this invention are directed to treating, slowing the progression, lessening the decline, delaying onset of symptoms or slowing the progression of symptoms of dyskinesia comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

Dyskinesias are abnormal, involuntary movements which may appear as jerking, twisting or writhing of parts of the body. There are several different types of dyskinesias, which can be categorized as chorea, dystonia, myoclonus, tremor and paroxysmal tardive (late-onset type). These movement disorders include, without limitation, parkinsonism, tardive dyskinesia, chorea, dystonia, tremor, akathisia, athetosis, myoclonus or tics. In some embodiments, the dyskinesia is L-DOPA Induced Dyskinesia (LID). In some embodiments, the dyskinesia is Parkinson's disease (PD)-LID.

In other embodiments, the methods of this invention are directed to treating, slowing the progression, lessening the decline, delaying onset of symptoms or slowing the progression of symptoms lessening the decline of Alzheimer's Disease comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

In other embodiments, the methods of this invention are directed to treating, slowing the progression, lessening the decline, delaying onset of symptoms or slowing the progression of symptoms age related memory loss comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

In other embodiments, the methods of this invention are directed to treating, slowing the progression, lessening the decline, delaying onset of symptoms or slowing the progression of symptoms of neurodegenerative eye disease, optic neuropathies including glaucoma, age-related macular degeneration (AMD), Leber's Hereditary Optic Neuropathy (LHON) geographic atrophy (GA), and retinitis pigmentosa, and related symptoms comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 1 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

In other embodiments, the methods of this invention are directed to treating, slowing the progression, lessening the decline, delaying onset of symptoms or slowing the progression of symptoms of Wolfram Disease comprising administering a composition comprising pridopidine or a pharmaceutically acceptable salt thereof and at least one of Compounds 1-8 or pharmaceutically acceptable salt thereof. In other embodiments Wolfram Disease symptoms comprise urinary tract abnormalities, ataxia, loss of sense of smell, loss of gag reflex, myoclonus, peripheral neuropathy, seizures, depression, impulsive and/or aggressive behavior, psychosis, gastrointestinal problems, intellectual disability, irregular breathing, central apnea, central respiratory failure, hypogonadism in males, stomach and/or intestinal ulcers, and a tendency to bleed excessively from wounds. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

In other embodiments, the methods of this invention are directed to treating, slowing the progression, lessening the decline, delaying onset of symptoms or slowing the progression of symptoms of bacterial infection-induced depression comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In another embodiment, the composition comprises a pridopidine salt.

In other embodiments, the methods of this invention are directed to treating, slowing the progression, lessening the decline, delaying onset of symptoms or slowing the progression of symptoms of Microphthalmia, syndromic 12 (MCOPS12), comprising administering a composition comprising pridopidine or a pharmaceutically acceptable salt thereof and at least one of Compounds 1-8 or pharmaceutically acceptable salt thereof. In another embodiment, the composition comprises pridopidine or a pharmaceutically acceptable salt thereof and at least one of compound 1, compound 4, pharmaceutically acceptable salt thereof or combination thereof. In another embodiment the composition comprises pridopidine or a pharmaceutically acceptable salt thereof and compound 1 or pharmaceutically acceptable salt thereof. In another embodiment the composition comprises pridopidine or a pharmaceutically acceptable salt thereof and compound 4 or pharmaceutically acceptable salt thereof. In another embodiment, the composition comprises pridopidine or a pharmaceutically acceptable salt thereof, compound 1 and compound 4 or pharmaceutically acceptable salt thereof. In other embodiment, the composition comprises a pridopidine base. In other embodiment, the composition comprises a pridopidine salt.

The subject invention further provides a method of treating, reducing or inhibiting a neurodegenerative eye disease or symptoms thereof in a subject comprising administering to the subject a composition comprising pridopidine or pharmaceutically acceptable salt thereof and at least one of compounds 1-8 or pharmaceutically acceptable salt thereof; wherein compounds 1-8 are represented by the following structures:

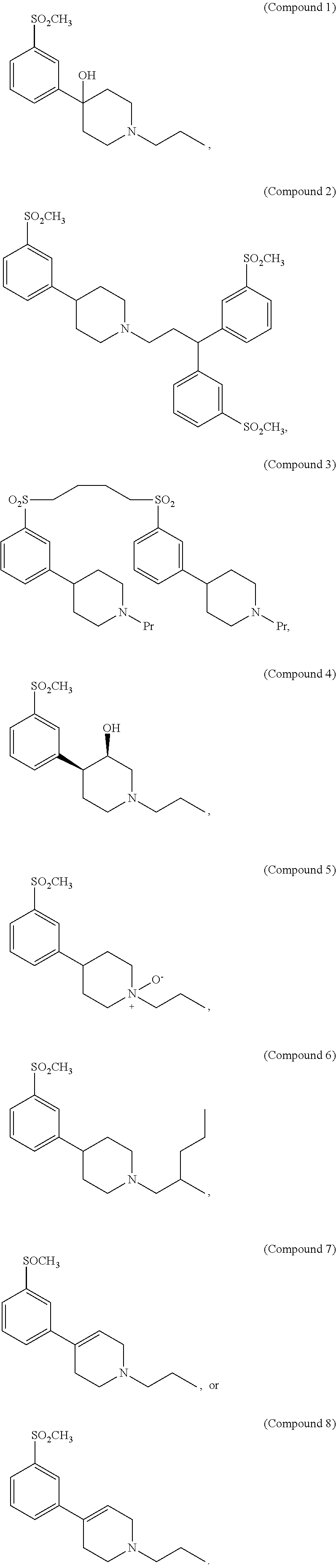

In other embodiment, the composition comprises a pridopidine base. In other embodiment, the composition comprises a pridopidine salt.

In other embodiment, the composition comprises pridopidine or a pharmaceutically acceptable salt thereof, Compound 1 or pharmaceutically acceptable salt thereof and Compound 4 or pharmaceutically acceptable salt thereof. In other embodiment, the composition comprises pridopidine or a pharmaceutically acceptable salt thereof, and at least one of Compound 1 and Compound 4 or pharmaceutically acceptable salts thereof. In other embodiment, the composition comprises pridopidine or a pharmaceutically acceptable salt thereof, and Compound 1 or pharmaceutically acceptable salt thereof. In other embodiment, the composition comprises pridopidine or a pharmaceutically acceptable salt thereof, and Compound 4 or pharmaceutically acceptable salt thereof.

The subject invention further provides a method of treating a subject afflicted with a neurodegenerative eye disease comprising administering to the subject a pharmaceutical composition comprising pridopidine or a pharmaceutically acceptable salt thereof and at least one of compounds 1-8 as described hereinabove, or pharmaceutically acceptable salt thereof effective to treat the subject. In other embodiment, the composition comprises pridopidine or a pharmaceutically acceptable salt thereof, Compound 1 or pharmaceutically acceptable salt thereof and Compound 4 or pharmaceutically acceptable salt thereof. In other embodiment, the composition comprises pridopidine or a pharmaceutically acceptable salt thereof, and at least one of Compound 1 and Compound 4 or pharmaceutically acceptable salts thereof. In other embodiment, the composition comprises pridopidine or a pharmaceutically acceptable salt thereof, and Compound 1 or pharmaceutically acceptable salt thereof. In other embodiment, the composition comprises pridopidine or a pharmaceutically acceptable salt thereof, and Compound 4 or pharmaceutically acceptable salt thereof.

The subject invention further provides a method of treating, reducing, or inhibiting a symptom of neurodegenerative eye disease in a subject afflicted with a neurodegenerative eye disease comprising administering to the subject a composition comprising pridopidine or pharmaceutically acceptable salt thereof.

The subject invention further provides a method of treating, reducing, or inhibiting a symptom of neurodegenerative eye disease in a subject afflicted with a neurodegenerative eye disease comprising administering to the subject a composition comprising pridopidine or pharmaceutically acceptable salt thereof and at least one of compounds 1-8 or pharmaceutically acceptable salt thereof. In other embodiment, the composition comprises pridopidine or a pharmaceutically acceptable salt thereof, Compound 1 or pharmaceutically acceptable salt thereof and Compound 4 or pharmaceutically acceptable salt thereof. In other embodiment, the composition comprises pridopidine or a pharmaceutically acceptable salt thereof, and at least one of Compound 1 and Compound 4 or pharmaceutically acceptable salts thereof. In other embodiment, the composition comprises pridopidine or a pharmaceutically acceptable salt thereof, and Compound 1 or pharmaceutically acceptable salt thereof. In other embodiment, the composition comprises pridopidine or a pharmaceutically acceptable salt thereof, and Compound 4 or pharmaceutically acceptable salt thereof.

In other embodiments the symptom of the neurodegenerative eye disease is optic nerve axon damage or loss. In other embodiments, the symptom is retinal ganglion cell (RGC) loss or death. In other embodiments, the symptom is a retinal pigment epithelial (RPE) cell loss or death. In other embodiments, the symptom is an optic nerve head astrocyte (ONHA) loss or damage. In other embodiments, the symptom is photoreceptor loss or death. In other embodiments, the composition is effective to reduce or prevent optic nerve axon loss or damage in a subject. In other embodiments, the composition is effective to reduce or prevent a retinal ganglion cell (RGC) loss or death in a subject. In other embodiments, the composition is effective to reduce or prevent a retinal pigment epithelium (RPE) cell loss or death in a subject. In other embodiments, the composition is effective to reduce or prevent photoreceptor loss or death in a subject. In other embodiments, the composition is effective to reduce or prevent an optic nerve head astrocyte (ONHA) loss or death in a subject.

In other embodiments, the optic nerve axon loss is reduced by at least 3%, by at least 5%, by at least 10%, by at least 20%, by at least 30%, by at least 40% or by at least 50%. In other embodiments, the optic nerve axon loss is reduced by more than 50%, more than 60%, more than 70%, or more than 80%. In other embodiments, the composition is effective to protect an optic nerve axon from degeneration in the subject. In other embodiments, the axon degeneration is induced by elevated intraocular pressure.

In other embodiments, the optic nerve head astrocyte (ONHA) loss is reduced by at least 3%, by at least 5%, by at least 10%, by at least 20%, by at least 25%, by at least 30%, by at least 35%, by at least 40% or by at least 50%. In other embodiments, the optic nerve axon loss is reduced by more than 50%, more than 60%, more than 70%, or more than 80%. In other embodiments, the composition is effective to protect an optic nerve head astrocyte (ONHA) from degeneration in the subject. In other embodiments, the astrocyte degeneration is induced by oxidative stress.

In one embodiment, the administration of a composition comprising pridopidine or pharmaceutically acceptable salt thereof and at least one of compounds 1-8 or pharmaceutically acceptable salt thereof is effective to reduce or inhibit a symptom of the neurodegenerative eye disease in the subject.

In an embodiment, the neurodegenerative eye disease is selected from the group consisting of glaucoma, Age-related Macular Degeneration (AMD), geographic atrophy (GA), optic neuropathy, Microphthalmia, syndromic 12 (MCOPS12) and retinitis pigmentosa. In another embodiment, the neurodegenerative eye disease refers to any disease affecting retinal ganglion cells, photoreceptors, other retinal neurons, and corneal nerves.

Diseases affecting retinal ganglion cells and their connections are optic neuropathies, and include glaucomatous optic neuropathy, also called glaucoma; inflammatory optic neuropathy, also called optic neuritis; ischemic optic neuropathy; toxic optic neuropathy; compressive optic neuropathy; infiltrative optic neuropathy; hereditary optic neuropathy; traumatic optic neuropathy; nutritional optic neuropathy; optic neuropathy from increased intracranial pressure, also called papilledema optic neuropathy; disc drusen optic neuropathy; autoimmune optic neuropathies; and other optic neuropathies. Each category of optic neuropathies may include subcategories, for example for ischemic optic neuropathy there is nonarteritic anterior ischemic optic neuropathy, arteritic anterior ischemic optic neuropathy, and posterior ischemic optic neuropathy.

In some embodiments, the neurodegenerative eye disease is glaucoma, including all clinical forms of glaucoma. For example, for glaucoma there is open-angle glaucoma and angle-closure glaucoma, and for each of those, there are sub-subcategories, for example, for open-angle glaucoma there is primary open-angle-glaucoma, pigmentary glaucoma, pseudoexfoliative glaucoma, neovascular glaucoma, steroid-induced glaucoma, normal-tension glaucoma, pressure-independent glaucoma, and many others.

The subject invention further provides a method of treating, reducing, or inhibiting glaucoma or a symptom thereof in a subject afflicted with glaucoma, comprising administering to the subject a composition comprising pridopidine or pharmaceutically acceptable salt thereof and at least one of compounds 1-8 or pharmaceutically acceptable salt thereof. In other embodiment, the composition comprises pridopidine or a pharmaceutically acceptable salt thereof, Compound 1 or pharmaceutically acceptable salt thereof and Compound 4 or pharmaceutically acceptable salt thereof. In other embodiment, the composition comprises pridopidine or a pharmaceutically acceptable salt thereof, and at least one of Compound 1 and Compound 4 or pharmaceutically acceptable salts thereof. In other embodiment, the composition comprises pridopidine or a pharmaceutically acceptable salt thereof, and Compound 1 or pharmaceutically acceptable salt thereof. In other embodiment, the composition comprises pridopidine or a pharmaceutically acceptable salt thereof, and Compound 4 or pharmaceutically acceptable salt thereof. In some embodiments, the glaucoma is: open-angle glaucoma, angle-closure glaucoma, primary open-angle-glaucoma, pigmentary glaucoma, pseudoexfoliative glaucoma, neovascular glaucoma, steroid-induced glaucoma, normal-tension glaucoma, or pressure-independent glaucoma; each represents a separate embodiment according to this invention.

Diseases affecting photoreceptors and other cells in the retina other than retinal ganglion cells include but not limited to: age-related macular degeneration (AMD), including wet and dry AMD; cystoid macular edema; central serous chorioretinopathy; macular pucker or macular hole; diabetic and nondiabetic macular edema; epiretinal membrane; all variants of retinitis pigmentosa and similar inherited or non-inherited retinal degenerations; retinal detachment; solar retinopathy; autoimmune retinopathy; retinal artery occlusions; retinal vein occlusions; diabetic retinopathy; infectious retinopathies; inflammation affecting the retina, including uveitis; degenerative retinal disorders from myopia; lattice degeneration.

Diseases affecting corneal nerves include but not limited to: infections, for example herpes viruses, leprosy, acanthamoeba, and fungi; toxic agents, for example topical anesthetics, preservative agents, and others; sensory neuropathies, for example trigeminal nerve disease or injury, hereditary or acquired polyneuropathies; corneal disease, for example corneal dystrophies, keratoconus, bullous keratopathy, and others; autoimmune diseases, for example Sjogren's syndrome; dry eyes; the effects of corneal surgery, for example after laser in situ keratomileusis (LASIK), corneal transplant, and others.

In one embodiment, the neurodegenerative eye disease is glaucoma. In another embodiment, the neurodegenerative eye disease is Wet Age-related Macular Degeneration (“Wet AMD”), Dry Age-related Macular Degeneration (“Dry AMD”) or geographic atrophy (GA). In a further embodiment, the neurodegenerative eye disease is Leber hereditary optic neuropathy (LHON).

In one embodiment, the symptom is retinal ganglion cell damage or retinal ganglion cell loss or optic nerve axon loss or damage. In another embodiment, the symptom is macular degeneration or retinal pigment epithelium (RPE) cell loss or damage.

In one embodiment, the method comprises reducing retinal ganglion cell loss or damage in the subject.

In one embodiment, the amount of pridopidine or pharmaceutically acceptable salt thereof and at least one of compounds 1-8 or pharmaceutically acceptable salt thereof is effective in reducing or preventing retinal ganglion cell loss or damage in the subject. In another embodiment, the retinal ganglion cell loss is reduced by at least 3%, at least 5%, at least 10%, by at least 20%, by at least 30%, by at least 40% or by at least 50%. In a further embodiment, the retinal ganglion cell loss is reduced by more than 50%, more than 60%, more than 70%, or more than 80%.

In one embodiment, the amount of pridopidine is effective in reducing or preventing retinal epithelium (RPE) cell loss or damage in the subject. In another embodiment, the RPE cell loss is reduced by at least 3%, at least 5%, at least 10%, by at least 20%, by at least 30%, by at least 40% or by at least 50%. In a further embodiment, the RPE cell loss is reduced by more than 50%, more than 60%, more than 70%, or more than 80%.

In one embodiment, the amount of pridopidine or pharmaceutically acceptable salt thereof and at least one of compounds 1-8 or pharmaceutically acceptable salt thereof is effective in improving retinal ganglion cell (RGC) viability in the subject. In another embodiment, the viability is improved by at least 3%, at least 5%, at least 10%, by at least 20%, by at least 23%, by at least 25%, by at least 30%, by at least 40%, by at least 45%, or by at least 50%.

In one embodiment, the amount of pridopidine is effective in reducing or preventing photoreceptor loss or damage in the subject. In another embodiment, the photoreceptor loss is reduced by at least 3%, at least 5%, at least 10%, by at least 20%, by at least 30%, by at least 40% or by at least 50%. In a further embodiment, the photoreceptor loss is reduced by more than 50%, more than 60%, more than 70%, or more than 80%.

In one embodiment, the amount of pridopidine is effective in reducing or preventing optic nerve head astrocyte (ONHA) loss or damage in the subject. In another embodiment, the optic nerve head astrocyte (ONHA) loss is reduced by at least 3%, at least 5%, at least 10%, by at least 20%, by at least 30%, by at least 40% or by at least 50%. In a further embodiment, the optic nerve head astrocyte (ONHA) loss is reduced by more than 50%, more than 60%, more than 70%, or more than 80%.

In one embodiment, the amount of pridopidine or pharmaceutically acceptable salt thereof and at least one of compounds 1-8 or pharmaceutically acceptable salt thereof is effective in reducing or preventing optic nerve head astrocytes (ONHAs) loss or damage in the subject. In another embodiment, the ONHAs loss is reduced by at least 3%, at least 5%, at least 10%, by at least 20%, by at least 23%, by at least 25%, by at least 30%, by at least 40%, by at least 45%, or by at least 50%.

In one embodiment, the amount of pridopidine or pharmaceutically acceptable salt thereof and at least one of compounds 1-8 or pharmaceutically acceptable salt thereof is effective in reducing oxidative stress in ONHAs in the subject. In another embodiment, the oxidative stress in ONHAs is reduced by at least 3%, at least 5%, at least 10%, by at least 20%, by at least 23%, by at least 25%, by at least 30%, by at least 33%, by at least 35%, by at least 40%, by at least 45%, or by at least 50%. In some embodiments, at least one of compounds 1-8 is compound 1. In some embodiments, at least one of compounds 1-8 is compound 4. In some embodiments, at least one of compounds 1-8 is compound 1 and compound 4.

In one embodiment, treatment comprises improving retinal ganglion cell viability in the patient by more than 50%, more than 60%, more than 70%, or more than 80%.

In one embodiment, treatment comprises improving RPE cell viability in the patient by more than 50%, more than 60%, more than 70%, or more than 80%.

In one embodiment, treatment comprises improving photoreceptor viability in the patient by more than 50%, more than 60%, more than 70%, or more than 80%.

In one embodiment, treatment comprises improving optic nerve head astrocytes (ONHAs) viability in the patient by more than 15%, more than 20%, more than 25%, or more than 35%.

In another embodiment, treatment comprises reducing retinal ganglion cell loss in the patient by more than 50%, more than 60%, more than 70%, or more than 80%.

In another embodiment, treatment comprises reducing RPE cell loss in the patient by more than 50%, more than 60%, more than 70%, or more than 80%.

In another embodiment, treatment comprises reducing photoreceptor loss in the patient by more than 50%, more than 60%, more than 70%, or more than 80%.

The subject invention also provides a method of preventing or reducing retinal ganglion cell (RGC) damage or loss in a subject, comprising administering to the subject a pharmaceutical composition comprising pridopidine or a pharmaceutically acceptable salt thereof and at least one of compounds 1-8 or pharmaceutically acceptable salt thereof effective to prevent or reduce retinal ganglion cell damage or loss in the subject. In one embodiment, the composition is effective to improve retinal ganglion cell viability in a subject. In another embodiment, the composition is effective to protect a retinal ganglion cell from cell death in the subject. In some embodiments, the cell death is induced by elevated intraocular pressure. In some embodiments, at least one of compounds 1-8 is compound 1. In some embodiments, at least one of compounds 1-8 is compound 4. In some embodiments, at least one of compounds 1-8 is compound 1 and compound 4.

The subject invention also provides a method of preventing or reducing retinal pigment epithelium (RPE) cell damage or loss in a subject, comprising administering to the subject a pharmaceutical composition comprising pridopidine or a pharmaceutically acceptable salt thereof and at least one of compounds 1-8 or pharmaceutically acceptable salt thereof effective to prevent or reduce retinal pigment epithelium (RPE) cell damage or loss in the subject. In one embodiment, the composition is effective to improve retinal pigment epithelium (RPE) cell viability in a subject. In another embodiment, the composition is effective to protect a retinal pigment epithelium cell from cell death in the subject. In some embodiments, the cell death is induced by elevated intraocular pressure. In some embodiments, at least one of compounds 1-8 is compound 1. In some embodiments, at least one of compounds 1-8 is compound 4. In some embodiments, at least one of compounds 1-8 is compound 1 and compound 4.

The subject invention also provides a method of preventing or reducing photoreceptor cell damage or loss in a subject, comprising administering to the subject a pharmaceutical composition comprising pridopidine or a pharmaceutically acceptable salt thereof and at least one of compounds 1-8 or pharmaceutically acceptable salt thereof effective to prevent or reduce photoreceptor cell damage or loss in the subject. In one embodiment, the composition is effective to improve photoreceptor cell viability in a subject. In another embodiment, the composition is effective to protect a photoreceptor cell from cell death in the subject. In some embodiments, the cell death is induced by elevated intraocular pressure. In some embodiments, at least one of compounds 1-8 is compound 1. In some embodiments, at least one of compounds 1-8 is compound 4. In some embodiments, at least one of compounds 1-8 is compound 1 and compound 4.

In one embodiment, the method comprises reducing optic nerve axon loss or damage in the subject.

In one embodiment, the pharmaceutical composition comprising pridopidine or a pharmaceutically acceptable salt thereof and at least one of compounds 1-8 or pharmaceutically acceptable salt thereof, is effective to reduce or prevent optic nerve axon loss or damage in the subject. In another embodiment, the optic nerve axon loss is reduced by at least 3%, at least 5%, at least 10%, by at least 20%, by at least 30%, by at least 40% or by at least 50%. In a further embodiment, the optic nerve axon loss is reduced by more than 50%, more than 60%, more than 70%, or more than 80%.

In one embodiment, treatment comprises improving optic nerve axon viability in the patient by more than 10%, more than 20%, more than 30%, more than 40%, more than 50%, more than 60%, more than 70%, or more than 80%.

In another embodiment, treatment comprises reducing optic nerve axon loss in the patient by more than 10%, more than 20%, more than 30%, more than 40%, more than 50%, more than 60%, more than 70%, or more than 80%.

The subject invention also provides a method of preventing or reducing optic nerve axon damage or loss in a subject, comprising administering to the subject a pharmaceutical composition comprising pridopidine or a pharmaceutically acceptable salt thereof effective to prevent or reduce optic nerve axon damage or loss in the subject. In one embodiment, the composition is effective to improve optic nerve axon viability in a subject. In another embodiment, the composition is effective to protect an optic nerve axon from cell death in the subject. In some embodiments, the cell death is induced by elevated intraocular pressure.

The subject invention also provides a method of preventing or reducing optic nerve axon damage or loss in a subject, comprising administering to the subject a pharmaceutical composition comprising pridopidine or a pharmaceutically acceptable salt thereof and at least one of compounds 1-8 or pharmaceutically acceptable salt thereof effective to prevent or reduce optic nerve axon damage or loss in the subject. In one embodiment, the composition is effective to improve optic nerve axon viability in a subject. In another embodiment, the composition is effective to protect an optic nerve axon from cell death in the subject. In some embodiments, the cell death is induced by elevated intraocular pressure. In some embodiments, at least one of compounds 1-8 is compound 1. In some embodiments, at least one of compounds 1-8 is compound 4. In some embodiments, at least one of compounds 1-8 is compound 1 and compound 4.

In one embodiment, the method comprises reducing optic nerve head astrocytes (ONHAs) loss or damage in the subject.

In one embodiment, the pharmaceutical composition comprising pridopidine or a pharmaceutically acceptable salt thereof and at least one of compounds 1-8 or pharmaceutically acceptable salt thereof, is effective to reduce or prevent optic nerve head astrocytes (ONHAs) loss or damage in the subject. In another embodiment, the optic nerve head astrocytes (ONHAs) loss is reduced by at least 3%, at least 5%, at least 10%, by at least 20%, by at least 30%, by at least 40% or by at least 50%. In a further embodiment, the optic nerve head astrocytes (ONHAs) loss is reduced by more than 50%, more than 60%, more than 70%, or more than 80%.

In one embodiment, treatment comprises improving optic nerve head astrocytes (ONHAs) viability in the patient by more than 10%, more than 20%, more than 30%, more than 40%, more than 50%, more than 60%, more than 70%, or more than 80%.

In another embodiment, treatment comprises reducing optic nerve head astrocytes (ONHAs) loss in the patient by more than 10%, more than 20%, more than 30%, more than 40%, more than 50%, more than 60%, more than 70%, or more than 80%.

The subject invention also provides a method of preventing or reducing optic nerve head astrocytes (ONHAs) damage or loss in a subject, comprising administering to the subject a pharmaceutical composition comprising pridopidine or a pharmaceutically acceptable salt thereof effective to prevent or reduce optic nerve head astrocytes (ONHAs) damage or loss in the subject. In one embodiment, the composition is effective to improve optic nerve head astrocytes (ONHAs) viability in a subject. In another embodiment, the composition is effective to protect an optic nerve head astrocyte (ONHA) from cell death in the subject. In some embodiments, the cell death is induced by oxidative stress.

The subject invention also provides a method of preventing or reducing optic nerve head astrocytes (ONHAs) damage or loss in a subject, comprising administering to the subject a pharmaceutical composition comprising pridopidine or a pharmaceutically acceptable salt thereof and at least one of compounds 1-8 or pharmaceutically acceptable salt thereof effective to prevent or reduce optic nerve head astrocytes (ONHAs) damage or loss in the subject. In one embodiment, the composition is effective to improve optic nerve head astrocytes (ONHAs) viability in a subject. In another embodiment, the composition is effective to protect an optic nerve head astrocyte (ONHA) from cell death in the subject. In some embodiments, the cell death is induced by oxidative stress. In some embodiments, at least one of compounds 1-8 is compound 1. In some embodiments, at least one of compounds 1-8 is compound 4. In some embodiments, at least one of compounds 1-8 is compound 1 and compound 4.

In another embodiment, treating comprises slowing progression of the neurodegenerative disease of the eye in the subject. In some embodiments, the treating comprises slowing progression of visual field loss towards blindness in a patient afflicted with glaucoma. In some embodiments, treating comprises preventing blindness in a patient afflicted with glaucoma.

In one embodiment, pridopidine is pridopidine hydrochloride.

For the methods and use disclosed herein, the route of administration can be, e.g., oral. Routes of administration can also be classified by whether the effect is local (e.g., in topical administration) or systemic (e.g., in enteral or parenteral administration). “Local administration” as used herein shall mean administration of a compound or composition directly to where its action is desired, and specifically excludes systemic administration. “Topical administration” of a compound or composition as used herein shall mean application of the compound or composition to body surfaces such as the skin or mucous membranes such as eyes. “Ocular administration” as used herein shall mean application of a compound or composition to the eye of a subject or to the skin around the eye (periocular skin) or the mucosa around the eye, specifically the conjunctiva of a subject, i.e., local administration. Examples of ocular administration include topical administration directly to the eye, topical application to the eye lid or injection into a portion of the eye or eye socket. In addition, an “ocular pharmaceutical composition” as used herein means a pharmaceutical composition formulated for ocular administration. The amount of pridopidine and the pharmaceutical compositions of the present invention may be administered by oral administration, topical administration, systemic administration, local administration, or ocular administration. In other embodiments, the composition described herein are administered orally, topically, intraocularly, periocularly or ocularly. In other embodiments, the composition described herein is administered by an eye drop application to the conjunctiva.

In one embodiment, the pharmaceutical composition comprising pridopidine or a pharmaceutically acceptable salt thereof is administered via systemic administration. In some embodiments, the pharmaceutical composition is administered via oral administration. In another embodiment, the pharmaceutical composition is administered in the form of an aerosol, an inhalable powder, an injectable, a liquid, a gel, a cream, a solid, a capsule or a tablet. In other embodiments, the composition described herein is administered orally, topically, intravitreally, intraocularly, periocularly or ocularly. In other embodiments, the composition described herein is administered by an eye drop application to the conjunctiva.

In one embodiment, the pharmaceutical composition comprising pridopidine or a pharmaceutically acceptable salt thereof and at least one of compounds 1-8 or pharmaceutically acceptable salt thereof is administered via systemic administration. In some embodiments, the pharmaceutical composition is administered via oral administration. In another embodiment, the pharmaceutical composition is administered in the form of an aerosol, an inhalable powder, an injectable, a liquid, a gel, a cream, a solid, a capsule or a tablet. In other embodiments, the composition described herein is administered orally, topically, intraocularly, intravitreally, periocularly or ocularly. In other embodiments, the composition described herein is administered by an eye drop application to the conjunctiva.

In one embodiment, the pharmaceutical composition described herein is administered via local administration to the eye. In another embodiment, the pridopidine and at least one of compounds 1-8 or pharmaceutically acceptable salt thereof is administered via topical administration. In a further embodiment, the pridopidine is administered via intraocular, periocular, or ocular administration. In some embodiments, the pridopidine is administered in the form of a liquid, a gel, a cream or a contact lens.

In another embodiment, the pharmaceutical composition described herein is administered directly to the eye of a subject, for example as eye drops, an intraocular depot injection, eye gels, a tablet inserted into the conjunctiva, or a lens loaded with pridopidine. In an embodiment, pridopidine hydrochloride is administered to the eye of the subject.

In one embodiment, the pharmaceutical composition described herein is part of a formulation suitable to be administered by ocular drops. The ocular drops can be in the form of a liquid or a gel, preferably in the form of a liquid. When the pharmaceutical composition is administered topically in the form of a liquid or gel to the eye, a lower amount of pridopidine is required to produce the same clinical effect as systemic administration of pridopidine.

In one embodiment, the amount of pridopidine administered systemically is 22.5 mg/day-315 mg/day, 90 mg/day-315 mg/day, 90-250 mg/day, or 90-180 mg/day. In another embodiment, the amount of pridopidine administered is about 22.5 mg/day, about 45 mg/day, about 67.5 mg/day, about 90 mg/day, about 100 mg/day, about 112.5 mg/day, about 125 mg/day, about 135 mg/day, about 150 mg/day, about 180 mg/day, about 200 mg/day, about 225 mg/day, about 250 mg/day, or about 315 mg/day. In other embodiments, the composition comprising pridopidine or pharmaceutically acceptable salt thereof is administered in a daily dose comprising an amount of pridopidine between 22.5 mg/day-315 mg/day. In other embodiments, the composition comprising pridopidine or pharmaceutically acceptable salt thereof and at least one of compounds 1-8 or pharmaceutically acceptable salt thereof is administered in a daily dose comprising an amount of pridopidine between 22.5 mg/day-315 mg/day.

In one embodiment, the amount of pridopidine administered systemically in a dose is about 22.5 mg, about 45 mg, about 67.5 mg, about 90 mg, about 100 mg, about 112.5 mg, about 125 mg, about 135 mg, about 150 mg, about 180 mg, about 200 mg, about 250 mg, or about 315 mg.

In another embodiment, the pharmaceutical composition described herein is administered directly to the eye of a subject. In some embodiments, pharmaceutical composition is formulated for direct administration to the eye, for example topical administration to the eye, for example as eye drops, and the pridopidine is prepared in a dose range of 0.1 mg to 50 mg, or 0.2 mg to 20 mg.

In one embodiment, the amount of pridopidine administered locally is 0.1 mg/day-50 mg/day or 0.2 mg/day 20 mg/day. In another embodiment, the amount of pridopidine administered locally in a dose is 0.1 mg-50 mg or 0.2 mg 20 mg.

In one embodiment the pharmaceutical composition described herein is administered periodically.

In one embodiment, the pharmaceutical composition described herein is administered daily.

In another embodiment, the pharmaceutical composition described herein is administered more often than once daily or less often than once daily. In one embodiment, the pharmaceutical composition described herein is administered more often than once daily, for example twice or thrice daily. In another embodiment, the pharmaceutical composition described herein is administered less often than once daily, for example, every other day or weekly.

In one embodiment, the periodic administration of the pharmaceutical composition described herein continues for at least 3 days, more than 30 days, more than 42 days, 8 weeks or more, at least 12 weeks, at least 24 weeks, more than 24 weeks, or 6 months or more. In some embodiments, for example, in the treatment of a subject with glaucoma, the treatment is a chronic treatment, with periodic administration of the pharmaceutical composition described herein for more than 12 months, more than 18 months, more than 24 months.

In one embodiment, the subject is a human patient.

In one embodiment, the method further comprises the administration of a second agent for the treatment of the neurodegenerative eye disease. In another embodiment, the second agent is a p-adrenergic antagonist, adrenergic agonist, parasympathomimetic agonist prostaglandin analog, or carbonic anhydrase inhibitor.

In another embodiment, the second agent reduces elevated intraocular pressure in a subject. In a further embodiment, the second agent is a prostaglandin agonist, a beta blocker, a carbonic anhydrase inhibitor, an alpha agonist, or a combination thereof. In an additional embodiment, the second agent is latanoprost, bimatoprost, travoprost ophthalmic, unoprostone ophthalmic, tafluprost, Betaxolol ophthalmic, Carteolol, timolol, levobunolol, metipranolol, Dorzolamide, brinzolamide, acetazol amide, methazolamide, brimonidine, Apraclonidine, or a combination thereof. In one embodiment, the second agent is pegcetacoplan.

In one embodiment, the subject is administered a fixed-dose combination comprising the pharmaceutical composition described herein and the second agent.

In one embodiment, the package further comprising a second pharmaceutical composition comprising an amount of a second agent for the treatment of a neurodegenerative eye disease, wherein the instructions provide for use of the first and second pharmaceutical compositions together to treat a subject afflicted with a neurodegenerative eye disease.

In one embodiment, the amount of pridopidine and the amount of the second agent are prepared to be administered simultaneously, contemporaneously or concomitantly.

The subject invention also provides a pharmaceutical composition comprising pridopidine or a pharmaceutical acceptable salt thereof and at least one of compounds 1-8 or pharmaceutically acceptable salt thereof for treating a subject afflicted with a neurodegenerative eye disease.

In one embodiment, the pharmaceutical composition further comprises an amount of a second agent for the treatment of a neurodegenerative eye disease.

In one embodiment, the pharmaceutical composition comprising pridopidine or pharmaceutically acceptable salt thereof and at least one of compounds 1-8 or pharmaceutically acceptable salt thereof and the second agent are prepared to be administered simultaneously, contemporaneously or concomitantly.

The subject invention also provides a pharmaceutical composition comprising an amount of pridopidine or pharmaceutically acceptable salt thereof for use in treating a subject afflicted with a neurodegenerative eye disease as an add-on therapy or in combination with a second agent for the treatment of a neurodegenerative eye disease.

The subject invention also provides a pharmaceutical composition comprising pridopidine or pharmaceutically acceptable salt thereof and at least one of compounds 1-8 or pharmaceutically acceptable salt thereof, for use in a combination therapy together with a pharmaceutical composition comprising a second agent for the treatment of a neurodegenerative eye disease.

The subject invention also provides a pharmaceutical composition comprising an amount of pridopidine or pharmaceutically acceptable salt thereof and at least one of compounds 1-8 or pharmaceutically acceptable salt thereof for use in treating a subject afflicted with a neurodegenerative eye disease as an add-on therapy or in combination with a second agent for the treatment of a neurodegenerative eye disease.

In one embodiment, the amount of pridopidine in the pharmaceutical composition is about 22.5 mg, about 45 mg, about 67.5, mg, about 90 mg, about 100 mg, about 112.5 mg, about 125 mg, about 135 mg, about 150 mg, about 180 mg, about 200 mg, about 250 mg, or about 315 mg.

In one embodiment, the amount of pridopidine in the pharmaceutical composition is 0.1 mg to 50 mg, or 0.2 mg to 20 mg.

In one embodiment, the dose of pridopidine in the pharmaceutical composition is measured as amount of pridopidine per weight of the subject. In another embodiment, the dose is between 1-100 mg/kg. In another embodiment, the dose is between 1-10, 20-50 or 50-100 mg/kg. In another embodiment, the dose is 3, 10, 30 or 60 mg/kg. The subject invention also provides a pharmaceutical composition in a unit dosage form, useful in treating a subject afflicted with a neurodegenerative eye disease, which comprises an amount of pridopidine or pharmaceutically acceptable salt thereof, wherein the amount of said pridopidine in said composition is effective, upon administration to said subject of one or more of said unit dosage forms of said composition, to treat the subject.

The invention also provides an ocular pharmaceutical composition comprising an amount of pridopidine or pharmaceutically acceptable salt thereof and at least one of compounds 1-8 or pharmaceutically acceptable salt thereof, and a pharmaceutically acceptable excipient suitable for administration to the eye.

In one embodiment, the ocular pharmaceutical composition further comprising a second agent for the treatment of the neurodegenerative eye disease. In one embodiment, the second agent for the treatment of the neurodegenerative eye disease is an anti-glaucoma agent.

In another embodiment, the amount of pridopidine in the ocular pharmaceutical composition is 0.1 mg to 50 mg, or 0.2 mg to 20 mg.

In one embodiment, the ocular pharmaceutical composition is in the form of a liquid. In some embodiments, the concentration of pridopidine in the ocular pharmaceutical composition is from 0.0001 to 10.0 w/v %, 0.001 to 5 w/v %, 0.01 to 1 w/v %, 0.1% to 10 w/v %.

The invention also provides the ocular pharmaceutical composition for use in treating a neurodegenerative eye disease in a subject.

The invention further provides an eye drop comprising the pharmaceutical composition. The invention additionally provides a container comprising eye drops and the pharmaceutical composition.

The invention also provides an eye drop or a container comprising eye drops for use in the methods of this invention.

Further provided is pridopidine or a pharmaceutically acceptable salt thereof and at least one of compounds 1-8 or pharmaceutically acceptable salt thereof for use in treating a subject afflicted with a neurodegenerative eye disease.

Provided herein is pridopidine for the manufacture of a medicament for use in treating a subject afflicted with a neurodegenerative eye disease.

### Terms

As used herein, and unless stated otherwise, each of the following terms shall have the definition set forth below.

As used herein, “pridopidine” means pridopidine base or a pharmaceutically acceptable salt thereof, as well as derivatives, for example deuterium-enriched version of pridopidine and salts.

A “salt thereof” is a salt of the instant compounds which have been modified by making acid or base salts of the compounds. The term “pharmaceutically acceptable salt” in this respect, refers to the relatively non-toxic, inorganic and organic acid or base addition salts of compounds of the present invention. For example, one means of preparing such a salt is by treating a compound of the present invention with an inorganic base.

“A neurodegenerative eye disease” as used herein is a disease which involves degeneration of neurosensory cells in the eye and/or of the optic nerve, including specifically retinal cells and/or their axons. Neurosensory cells include retinal ganglion cells, optic nerve axon, retinal pigment epithelium cells, cones, rods, and all other neuronal or glial cell types of the retina. Neurodegenerative eye diseases are exemplified by glaucoma, age-related macular degeneration (AMD), including wet and dry AMD, all variants of retinitis pigmentosa, optic neuropathy, including but not limited to ischemic optic neuropathy (ION), hereditary Leber hereditary optic neuropathy (LHON), and retinopathies including for example Stargardt's retinopathy. Neurodegenerative eye diseases are exemplified by diseases of neurons of the eye and their connections, as exemplified by diseases affecting retinal ganglion cells, photoreceptors, other retinal neurons, and corneal nerves.

Diseases affecting retinal ganglion cells and their connections are optic neuropathies, and include glaucomatous optic neuropathy, also called glaucoma; inflammatory optic neuropathy, also called optic neuritis; ischemic optic neuropathy; toxic optic neuropathy; compressive optic neuropathy; infiltrative optic neuropathy; hereditary optic neuropathy; traumatic optic neuropathy; nutritional optic neuropathy; optic neuropathy from increased intracranial pressure, also called papilledema optic neuropathy; disc drusen optic neuropathy; autoimmune optic neuropathies; and other optic neuropathies. Each category of optic neuropathies may include subcategories, for example for ischemic optic neuropathy there is nonarteritic anterior ischemic optic neuropathy, arteritic anterior ischemic optic neuropathy, and posterior ischemic optic neuropathy.

In some embodiments, the neurodegenerative eye disease is glaucoma, including all clinical forms of glaucoma. For example, for glaucoma there is open-angle glaucoma and angle-closure glaucoma, and for each of those, there are sub-subcategories, for example, for open-angle glaucoma there is primary open-angle-glaucoma, pigmentary glaucoma, pseudoexfoliative glaucoma, neovascular glaucoma, steroid-induced glaucoma, normal-tension glaucoma, pressure-independent glaucoma, and many others. In some embodiments, the neurodegenerative eye disease is glaucoma, including all clinical forms of glaucoma, for example, primary glaucoma or secondary glaucoma. A primary glaucoma is for example, primary open angle glaucoma (POAG), normal-tension glaucoma (NTG), primary angle-closure glaucoma (PACG), acute angle-closure glaucoma (AACG) and angle-closure glaucoma (ACG). A secondary glaucoma is for example, pseudoexfoliation glaucoma, pigmentary glaucoma, neovascular glaucoma, steroid-induced glaucoma, and treatment refractory glaucoma.

Diseases affecting photoreceptors and other cells in the retina other than retinal ganglion cells include age-related macular degeneration (AMD), including wet and dry AMD; geographic atrophy (GA), cystoid macular edema; central serous chorioretinopathy; macular pucker or macular hole; diabetic and nondiabetic macular edema; epiretinal membrane; all variants of retinitis pigmentosa and similar inherited or non-inherited retinal degenerations; retinal detachment; solar retinopathy; autoimmune retinopathy; retinal artery occlusions; retinal vein occlusions; diabetic retinopathy; infectious retinopathies; inflammation affecting the retina, including uveitis; degenerative retinal disorders from myopia; lattice degeneration. In one embodiment, the disease is Microphthalmia, syndromic 12 (MCOPS12).

Diseases affecting corneal nerves include infections, for example herpes viruses, leprosy, acanthamoeba, and fungi; toxic agents, for example topical anesthetics, preservative agents, and others; sensory neuropathies, for example trigeminal nerve disease or injury, hereditary or acquired polyneuropathies; corneal disease, for example corneal dystrophies, keratoconus, bullous keratopathy, and others; autoimmune diseases, for example Sjogren's syndrome; dry eyes; the effects of corneal surgery, for example after laser in situ keratomileusis (LASIK), corneal transplant, and others.

As used herein, an “amount” or “dose” of pridopidine as measured in milligrams refers to the milligrams of pridopidine (4-[3-(methylsulfonyl)phenyl]-1-propyl-piperidine) present in a preparation, regardless of the form of the preparation. For example, a unit dose containing “90 mg pridopidine” means the amount of pridopidine in a preparation is 90 mg, regardless of the form of the preparation. Thus, when in the form of a salt, e.g. pridopidine hydrochloride, the weight of the salt form necessary to provide a dose of 90 mg pridopidine would he greater than 90 mg due to the presence of the salt.

As used herein, a “unit dose”, “unit doses” and “unit dosage form(s)” mean a single drug administration entity/entities.

As used herein, “about” in the context of a numerical value or range means ±10% of the numerical value or range recited or claimed.

As used herein, “effective” when referring to an amount of pridopidine refers to the quantity of pridopidine that is sufficient to yield a desired therapeutic response. Efficacy can be measured by e.g., a reduced retinal ganglion cell number or optic nerve axon loss or damage.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

As used herein, “a pharmaceutically acceptable excipient suitable for administration to the eye” includes any excipient that is known to be or expected to be suitable for administration directly to the eye.

Excipients (or additives) that are usually used in formulating ocular drops can be used together with pridopidine. Excipients may include preservatives, including quaternary ammonium salts such as benzalkonium chloride, benzethonium chloride and the like; cationic compounds such as chlorhexidine gluconate and the like; p-hydroxybenzoates such as methyl p-hydroxybenzoate, propyl p-hydroxybenzoate and the like; alcohol compounds such as chlorobutanol, benzyl alcohol and the like; sodium dehydroacetate; thimerosal; sorbic acid; and the like (U.S. Pat. No. 6,114,319). The formulation suitable to be administered by ocular drops may include a buffer, such as acetates such as sodium acetate and the like, phosphates such as sodium dihydrogenphosphate, disodium hydrogenphosphate, potassium dihydrogenphosphate, dipotassium hydrogenphosphate and the like, aminocaproic acid, amino acid salts such as sodium glutamate and the like, boric acid and salt thereof, citric acid and salt thereof, and the like (U.S. Pat. No. 6,114,319). The formulation suitable to be administered by ocular drops may include excipients, such as a stabilizer, an antioxidant, a pH adjusting agent, a chelating agent, a thickener and the like (U.S. Pat. No. 6,114,319). Examples of the antioxidant include ascorbic acid and salt thereof, sodium thiosulfate, sodium hydrogensulfite, tocopherol, sodium thiosulfate, sodium hydrogensulfite, pyruvic acid and salt thereof, and the like (U.S. Pat. No. 6,114,319). Examples of chelating agent include sodium edetate, citric acid and salt thereof, and the like (U.S. Pat. No. 6,114,319). Examples of the pH adjusting agent include hydrochloric acid, phosphoric acid, acetic acid, sodium hydroxide, sodium hydrogencarbonate, potassium hydroxide, sodium carbonate, sulfuric acid, aqueous ammonia and the like (U.S. Pat. No. 6,114,319). The pH of the formulation suitable for administration by ocular drops may be at any point within an ophthalmologically acceptable range, for example, between pH 5.0 and pH 8.0. When pridopidine is to be administered by ocular drops or eye drops, it is preferable to prepare the formulation so that the concentration of pridopidine is from 0.0001 to 10.0 w/v %.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of the invention. Examples of pharmaceutically acceptable salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Pharmaceutical Composition for Use in the Methods of this Invention:

In some embodiments the methods of this invention make use of a pharmaceutical composition comprising pridopidine or pharmaceutically acceptable salt thereof. In some embodiments the methods of this invention make use of a pharmaceutical composition comprising pridopidine or pharmaceutically acceptable salt thereof and at least one of compounds 1-8:

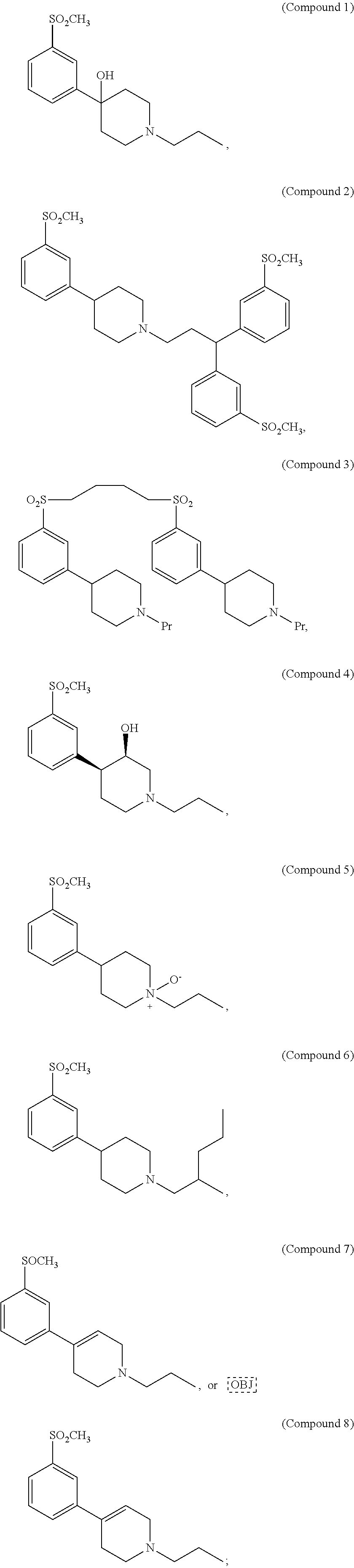

or pharmaceutically acceptable salt thereof.

In other embodiments the methods of this invention make use of a pharmaceutical composition comprising pridopidine or pharmaceutically acceptable salt thereof and compound 2 or pharmaceutically acceptable salt thereof.

In other embodiments the methods of this invention make use of a pharmaceutical composition comprising pridopidine or pharmaceutically acceptable salt thereof and compound 3 or pharmaceutically acceptable salt thereof.

In other embodiments the methods of this invention make use of a pharmaceutical composition comprising pridopidine or pharmaceutically acceptable salt thereof and compound 4 or pharmaceutically acceptable salt thereof.

In other embodiments the methods of this invention make use of a pharmaceutical composition comprising pridopidine or pharmaceutically acceptable salt thereof and compound 5 or pharmaceutically acceptable salt thereof.

In other embodiments the methods of this invention make use of a pharmaceutical composition comprising pridopidine or pharmaceutically acceptable salt thereof and compound 6 or pharmaceutically acceptable salt thereof.

In other embodiments the methods of this invention make use of a pharmaceutical composition comprising pridopidine or pharmaceutically acceptable salt thereof and compound 7 or pharmaceutically acceptable salt thereof.

In other embodiments the methods of this invention make use of a pharmaceutical composition comprising pridopidine or pharmaceutically acceptable salt thereof and compound 8 or pharmaceutically acceptable salt thereof.

In other embodiments the methods of this invention make use of a pharmaceutical composition comprising pridopidine or a pharmaceutically acceptable salt thereof and at least one of compound 1, compound 4, pharmaceutically acceptable salt thereof or any combination thereof.

In other embodiments the methods of this invention make use of a pharmaceutical composition comprising pridopidine or a pharmaceutically acceptable salt thereof and compound 1 or pharmaceutically acceptable salt thereof.

In other embodiments the methods of this invention make use of a pharmaceutical composition comprising pridopidine or a pharmaceutically acceptable salt thereof and compound 4 or pharmaceutically acceptable salt thereof.

In other embodiments the methods of this invention make use of a pharmaceutical composition comprising pridopidine or a pharmaceutically acceptable salt thereof, compound 1 and compound 4 or pharmaceutically acceptable salt thereof.

In other embodiments the methods of this invention make use of a pharmaceutical composition comprising pridopidine salt, wherein the salt is hydrochloride, hydrobromide, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methane-sulphonate, naphthalene-2-sulphonate, phthalate, salicylate, sorbate, stearate, succinate, tartrate or toluene-p-sulphonate salt.

In other embodiments the methods of this invention make use of a pharmaceutical composition comprising at least one of compounds 1-8 salt, wherein the salt is hydrochloride, hydrobromide, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methane-sulphonate, naphthalene-2-sulphonate, phthalate, salicylate, sorbate, stearate, succinate, tartrate or toluene-p-sulphonate salt.

In other embodiments the methods of this invention make use of a pharmaceutical composition comprising pridopidine or pharmaceutically acceptable salt thereof and at least one of compounds 1-8 or pharmaceutically acceptable salt thereof, wherein the weight ratio between the pridopidine and at least one of compounds 1-8 is in the range of 1:0.001 to 1:0.1. In other embodiments, the weight ratio between the pridopidine and at least one of compounds 1-8 is in the range of 1:0.005 to 1:0.1. In other embodiment, the weight ratio between the pridopidine and at least one of compounds 1-8 is in the range of 1:0.001 to 1:0.005.

In other embodiments the methods of this invention make use of a pharmaceutical composition comprising pridopidine or pharmaceutically acceptable salt thereof and at least one of compounds 1-8 or pharmaceutically acceptable salt thereof, wherein the weight ratio between the pridopidine and at least one of compounds 1-8 is in the range of 1:100 to 100:1. In other embodiment, the weight ratio between the pridopidine and at least one of compounds 1-8 is in the range of 1:50 to 50:1. In other embodiment, the weight ratio between the pridopidine and at least one of compounds 1-8 is in the range of 1:10 to 20:1. In other embodiment, the weight ratio between the pridopidine and at least one of compounds 1-8 is in the range of 1:5 to 10:1. In other embodiment, the weight ratio between the pridopidine and at least one of compounds 1-8 is in the range of 1:5 to 5:1. In other embodiment, the weight ratio between the pridopidine and at least one of compounds 1-8 is in the range of 1:3 to 7:1. In other embodiment, the weight ratio between the pridopidine and at least one of compounds 1-8 is in the range of 5:1 to 1:2. In other embodiment, the weight ratio between the pridopidine and at least one of compounds 1-8 is 1:2. In other embodiment, the weight ratio between the pridopidine and at least one of compounds 1-8 is 5:1. In some embodiments, at least one of compounds 1-8 is compound 1. In some embodiments, at least one of compounds 1-8 is compound 4. In some embodiments, at least one of compounds 1-8 is compound 1 and compound 4.

In other embodiment, the weight ratio between the pridopidine and compound 4 is in the range of 10:1 to 1:10. In other embodiment, the weight ratio between the pridopidine and compound 4 is in the range of 7:1 to 1:7. In other embodiment, the weight ratio between the pridopidine and compound 4 is in the range of 5:1 to 1:5. In other embodiment, the weight ratio between the pridopidine and compound 4 is in the range of 5:1 to 1:1. In other embodiment, the weight ratio between the pridopidine and compound 4 is in the range of 1:1 to 1:5. In other embodiment, the weight ratio between the pridopidine and compound 4 is in the range of 1:1 to 1:2. In other embodiment, the weight ratio between the pridopidine and compound 4 is 1:2.

In other embodiment, the weight ratio between the pridopidine and compound 1 is in the range of 10:1 to 1:10000. In other embodiment, the weight ratio between the pridopidine and compound 1 is in the range of 10:1 to 1:500. In other embodiment, the weight ratio between the pridopidine and compound 1 is in the range of 5:1 to 1:100. In other embodiment, the weight ratio between the pridopidine and compound 1 is in the range of 1:1 to 1:100. In other embodiment, the weight ratio between the pridopidine and compound 1 is 5:1.

In other embodiments, the concentration of compounds 1, 2, 3, 4, 5, 6, 7 or 8 or pharmaceutically acceptable salt thereof within the composition is between 0.001% w/w to 10% w/w. In other embodiments, the concentration of compounds 1, 2, 3, 4, 5, 6, 7 or 8 or pharmaceutically acceptable salt thereof within the composition is between 0.001% w/w to 0.05% w/w. In other embodiments, the concentration of compounds 1, 2, 3, 4, 5, 6, 7 or 8 or pharmaceutically acceptable salt thereof within the composition is between 0.05% w/w to 0.35% w/w. In other embodiments, the concentration of compounds 1, 2, 3, 4, 5, 6, 7 or 8 or pharmaceutically acceptable salt thereof within the composition is between 0.001% w/w to 0.5% w/w. In other embodiments, the concentration of compounds 1, 2, 3, 4, 5, 6, 7 or 8 or pharmaceutically acceptable salt thereof within the composition is between 0.001% w/w to 0.15% w/w. In other embodiments, the concentration of compounds 1, 2, 3, 4, 5, 6, 7 or 8 or pharmaceutically acceptable salt thereof within the composition is between 0.01% w/w to 0.15% w/w. In other embodiments, the concentration of compounds 1, 2, 3, 4, 5, 6, 7 or 8 or pharmaceutically acceptable salt thereof within the composition is between 0.01% w/w to 0.5% w/w. In other embodiments, the concentration of compounds 1, 2, 3, 4, 5, 6, 7 or 8 or pharmaceutically acceptable salt thereof within the composition is between 0.01% w/w to 1% w/w. In other embodiments, the concentration of compounds 1, 2, 3, 4, 5, 6, 7 or 8 or pharmaceutically acceptable salt thereof within the composition is between 10% w/w to 90% w/w. In other embodiments, the concentration of compounds 1, 2, 3, 4, 5, 6, 7 or 8 or pharmaceutically acceptable salt thereof within the composition is between 20% w/w to 60% w/w. In other embodiments, the concentration of compounds 1, 2, 3, 4, 5, 6, 7 or 8 or pharmaceutically acceptable salt thereof within the composition is between 25% w/w to 75% w/w. In other embodiments, the concentration of compounds 1, 2, 3, 4, 5, 6, 7 or 8 or pharmaceutically acceptable salt thereof within the composition is between 25% w/w to 50% w/w. In other embodiments, the concentration of compounds 1, 2, 3, 4, 5, 6, 7 or 8 or pharmaceutically acceptable salt thereof within the composition is between 10% w/w to 30% w/w. In other embodiments, the concentration of compounds 1, 2, 3, 4, 5, 6, 7 or 8 or pharmaceutically acceptable salt thereof within the composition is between 60% w/w to 85% w/w.

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries. In an embodiment, the invention provides pharmaceutical compositions comprising the active compounds or pharmaceutically acceptable salts or derivatives thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

In other embodiments, the composition described herein is administered orally, topically, intraocularly, intravitreally, periocularly or ocularly. In other embodiments, the composition described herein is administered by an eye drop application to the conjunctiva.

In other embodiments, the composition described herein is administered locally (e.g., in topical administration) or systemic (e.g., in enteral or parenteral administration).
“Local administration” as used herein refers to administration of the composition directly to where its action is desired, and specifically excludes systemic administration.
“Topical administration” as used herein refers to administration of the composition to body surfaces such as the skin or mucous membranes such as eyes.
“Ocular administration” as used herein refers to administration of the composition to the eye of a subject or to the skin around the eye (periocular skin) or the mucosa around the eye, specifically the conjunctiva of a subject, i.e., local administration. Examples of ocular administration include topical administration directly to the eye, topical application to the eye lid or injection into a portion of the eye or eye socket.

The composition described herein are administered orally, topically, intraocularly, intravitreally, periocularly, ocularly. In another embodiment, administered to the cornea, conjunctive, subconjunctival, subtenons, intracameral, intravitreal, subretinal, under the lid, retrobulbar.

In other embodiments, the composition described herein is administered by an eye drop application to the conjunctiva.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection.

The pharmaceutical composition for use in the methods of this invention is an oral dosage unit formulated as a tablet, a capsule, a pill, powder, liquid solution or as a liquid suspension.

In other embodiments, the composition described herein is formulated as eye drops, ophthalmic solutions, ophthalmic suspensions, ophthalmic emulsions, eye ointments, eye sprays.

In other embodiments, the pharmaceutical composition described herein may be in the forma of an ophthalmic composition for topical application to an eye of a subject. The term “ophthalmic composition” as used herein will be understood to refer to any composition specifically formulated for direct and local administration to an eye of a patient. Said composition may be formulated for topical administration to the eye or for injection into the eye (i.e., intravitreal or intraocular injection). The ophthalmic composition may be provided in any formulation that allows for local administration thereof to the eye and allows the therapeutic compounds to function in accordance with the present disclosure. For example, but not by way of limitation, the ophthalmic composition may be provided in the form of a solution drops, a mist/spray, plasters and pressure sensitive adhesives, an ointment, a lotion, a cream, a gel, lyophilized/spray-dried forms, and the like. In one particular nonlimiting embodiment, the ophthalmic composition is provided in a form for topical application, such as but not limited to, an eyedrop formulation. In addition, the ophthalmic compositions of the present disclosure may be designed to provide delayed, controlled, extended, and/or sustained release using formulation techniques which are well known in the art.

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In an embodiment, the invention provides pharmaceutical compositions comprising the active compounds or pharmaceutically acceptable salts or derivatives thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

“Treating” as used herein encompasses, e.g., inducing inhibition, regression, or stasis of a disease or disorder, e.g., glaucoma, or alleviating, lessening, suppressing, inhibiting, reducing the severity of, eliminating or substantially eliminating, or ameliorating a symptom of the disease or disorder. Treatment further comprises providing neuroprotection to an ocular cell, for example a retinal ganglion cell or optic nerve axon in a subject. The “neuroprotective” activity of pridopidine is disclosed herein. Neuroprotection comprises protection of neurons, for example RGC or optic nerve axon, from injury or death or b) improvement of neuronal function for example of RGC, RPE, photoreceptor, optic nerve head astrocyte (ONHA) or optic nerve axon. As used herein, “neuroprotection” refers to reducing, preventing, attenuating and/or reversing progression of neurodegeneration. As used herein, “neurodegeneration” refers to the progressive loss of neurons, for example RGC or optic nerve axon loss, by injury or death.

“Inhibition” of disease progression or disease complication in a subject means preventing or reducing the disease progression and/or disease complication in the subject.

A “symptom” associated with glaucoma includes any clinical or laboratory manifestation associated with glaucoma and is not limited to what the subject can feel or observe.

As used herein, a subject “afflicted” with glaucoma means the subject has been diagnosed with glaucoma.

As used herein, a subject at “baseline” is as subject prior to administration of pridopidine in a therapy as described herein.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1 mg-40.0 mg” includes 0.1 mg, 0.2 mg, 0.3 mg, 0.4 mg, etc. up to 40.0 mg.

As used herein, a “fixed-dose combination” or “fixed-dosage combination” refers to a medicament which comprises two active agents. Typically, the two agents are very difficult to separate by means readily available to patients. Non-limiting examples include tablets, pills, or solutions comprising two agents.

In this application, when a comparative term is used, such as “the retinal ganglion cell (or optic nerve axon, or retinal pigment epithelium cell, or photoreceptor cell or optic nerve head astrocytes) loss is reduced by at least 10% in a subject” the comparison is relative to a subject afflicted with an analogous disease for example the control subject in a prior relevant clinical study, and not to a healthy subject. For example, the retinal ganglion cell (or optic nerve axon, or retinal pigment epithelium cell, or photoreceptor cell or optic nerve head astrocytes) loss may be compared to the average retinal ganglion cell (or optic nerve axon, or retinal pigment epithelium cell, or photoreceptor cell or optic nerve head astrocytes) loss in similarly diseased subjects without treatment with pridopidine. Thus, the comparison value may be obtained by reference to the placebo group of a clinical study.

The combination of the invention may be formulated for its simultaneous, separate or sequential administration, with at least a pharmaceutically acceptable earner, additive, adjuvant or vehicle as described herein. Thus, the combination of the two active compounds may be administered:
as a combination that is part of the same medicament formulation, the two active compounds are then administered simultaneously, or
as a combination of two units, each with one of the active substances giving rise to the possibility of simultaneous, sequential or separate administration.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding pridopidine therapy to a glaucoma patient already receiving therapy with IOP reducing eye drops.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

This invention will be better understood by reference to the Experimental Details which follow, but those skilled in the art will readily appreciate that the specific experiments detailed are only illustrative of the invention as described more fully in the claims which follow thereafter.

### EXAMPLES

### Example 1: Synergistic Effect of Pridopidine and Compound 1 or Pridopidine and Compound 4

Compound 1 and Compound 4 both display a synergistic effect with pridopidine on BDNF secretion from B104 neuroblastoma cells.

Compound 1 and Compound 4 show selective binding to the Sigma-1 Receptor (SiR, Ki=0.37 μM for compound 1 and Ki=2.9 μM for compound 4) with no binding to the Sigma-2 receptor (S2R, Ki>100 μM for both compound 1 and 4), as shown in Table 1.

TABLE 1
Binding affinity of pridopidine, Compound 1 and Compound 4
to the Sigma-1 and Sigma-2 receptors

| | S1R Ki | S2R Ki | S1R fold selectivity |
|---|---|---|---|
| Compound | (μM) | (μM) | (S2R/S1R) |
| Pridopidine | 0.057 | 5.45 | 96 |
| Compound 1 | 0.37 | >100 | >270 |
| Compound 4 | 2.9 | >100 | >35 |

In-vitro binding assays performed at Eurofins Panlabs Taiwan, Ltd. Specific ligand binding was determined in the presence of an excess of unlabelled ligand. Inhibition constants (Ki) were calculated from in vitro binding assays using the Cheng Prusoff equation (Cheng and Prusoff 1973). Source: Johnston et al, 2019 (Johnston et al. 2019) and NC20-PHARM-2.

Thus, both Compound 1 and Compound 4 have high affinity to the SiR and no affinity (Ki>100) to the S2R.

Reductions in Brain-Derived Neurotrophic Factor (BDNF) levels play a key role in the pathogenesis of neurodegenerative disorders and its levels are reduced in neurodegenerative and neurodevelopmental disorders such as Huntington disease (HD), Parkinson's disease, Alzheimer's disease (Zuccato and Cattaneo 2009) and Rett syndrome (Katz 2014).

Pridopidine demonstrates a dose dependent increase in BDNF secretion in rat neuroblastoma cells using an in-situ ELISA assay. This effect is mediated by activation of SiR, since pharmacological inhibition of the SiR abolished pridopidine's effect (Geva, et al. 2016).

When assessing the effect of Compound 1 or Compound 4 with pridopidine, the applicant identified an unexpected synergistic effect. The effect was observed in a BDNF in-situ ELISA assay (Geva, et al. 2016).

Thus, the synergistic effect on BDNF release demonstrated below is directly relevant to the therapeutic effect of pridopidine and compound 1 and compound 4.

The following data surprisingly and unexpectedly show that pridopidine together with either Compound 4 or Compound 1 demonstrates a synergistic effect on BDNF release.

### Synergistic Effect of Compound 4 and Pridopidine on BDNF Release

Pridopidine alone induces an increase in BDNF release of +13.6% at a concentration of 0.001 μM and +26% at a concentration of 0.005 μM, compared to control untreated cells. Compound 4 at a concentration of 0.001 μM alone has no effect on BDNF release compared to untreated control cells (−1.5%). However, pridopidine and Compound 4 together have an unexpected synergistic effect on BDNF release.

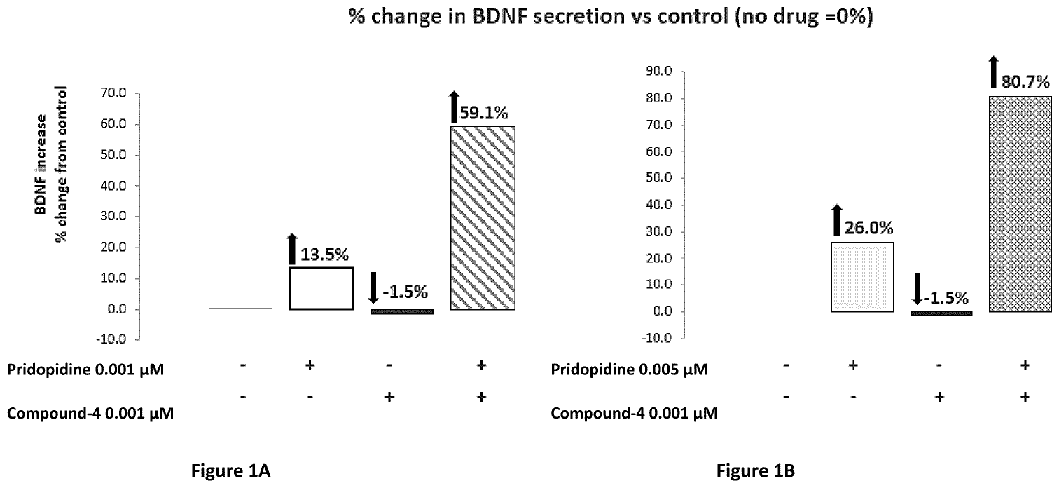

Pridopidine 0.001 μM+Compound 4 at 0.001 μM induce a 59.1% increase in BDNF release compared to control untreated cells (FIG. 1A).

Pridopidine 0.005 μM+Compound 4 at 0.001 μM induce an 80.7% increase in BDNF release compared to control untreated cells (FIG. 1B).

The effect of pridopidine and Compound 4 together is greater than the sum of the effects of each compound individually, indicating a surprising synergistic effect on BDNF secretion. The results are shown where the values are presented as percent (%) of change compared to untreated control.

### Synergistic Effect of Compound 1 and Pridopidine on BDNF Release

Pridopidine alone at a concentration of 0.01 μM induces an increase in BDNF release compared to control untreated cells of +3.4%. Compound 1 alone at a concentration of 1 μM induces a +12.5% increase in BDNF release compared to control. However, pridopidine and Compound 1 together have a synergistic effect on BDNF release (+53.1%).

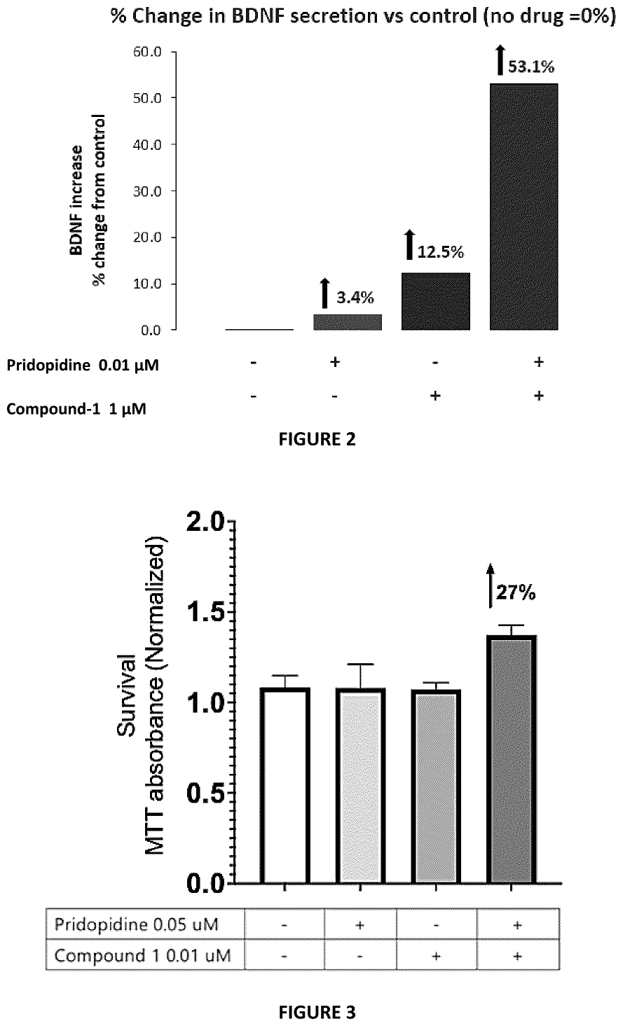

Pridopidine (0.01 μM)+Compound 1 (1 μM) induce a 53.1% increase in BDNF release compared to control untreated cells (FIG. 2).

Again, these results indicate a surprising and unexpected synergistic effect of pridopidine and Compound 1 on BDNF secretion as their effect when administered together (+53.1%) is greater than the sum of the effects of each compound individually.

Thus, the applicant has shown that Compounds 1 and Compound 4 have selective binding affinity to the SiR, together with a surprising and unexpected synergistic effect with pridopidine on BDNF release.

### Example 2: Combination Effect of Pridopidine and Compound 1 or Pridopidine and Compound 4 in Optic Nerve Head Astrocytes (ONHA)

Astrocytes form the majority of glia cells in the eye and optic nerve, and in glaucoma they become reactive, influencing the optic nerve head microenvironment and disease outcome. Exposure of optic nerve head astrocytes to oxidative stress can contribute to axon damage and additional hallmarks of glaucoma. Oxidative stress induced by tBHP has been shown to trigger distinct pathways of cellular damage in ONHAs, which lead to decreased cell function and degeneration of ONHAs and retinal ganglion cells (RGCs). Mitochondrial function is decreased in response to tBHP, resulting in an increase in reactive oxygen species (ROS) levels (increased oxidative stress).

The effects of pridopidine in combination with Compound 1 and Compound 4 against tBHP-induced oxidative stress in primary rat optic nerve head astrocytes (ONHA) were evaluated. Cellular oxidative stress was evaluated by measuring ROS levels using the cell-permeable ROS indicator chloromethyl derivative of 2′,7′-dichlorodihydrofluorescein diacetate (CM-H₂DCFDA). CM-H₂DCFDA is cleaved to form non-fluorescent H₂DCF, which is then oxidized to form the fluorescent DCF. DCF levels are indicative of ROS. Cell viability was also assessed using the commonly used colorimetric MTT assay.

### Pridopidine and Compound 1 Increase Cell Viability Following Oxidative Stress in a Synergistic Manner

In ONHA following oxidative stress with tBHP (40 μM), neither pridopidine (0.05 μM) nor compound 1 (0.01 μM) have any effect on cell viability when administered individually. In combination, pridopidine and compound 1 show an unexpected and surprising synergistic effect, increasing cell viability by 27%. (FIG. 3)

### Pridopidine and Compound 4 Increase Cell Viability Following Oxidative Stress in a Synergistic Manner

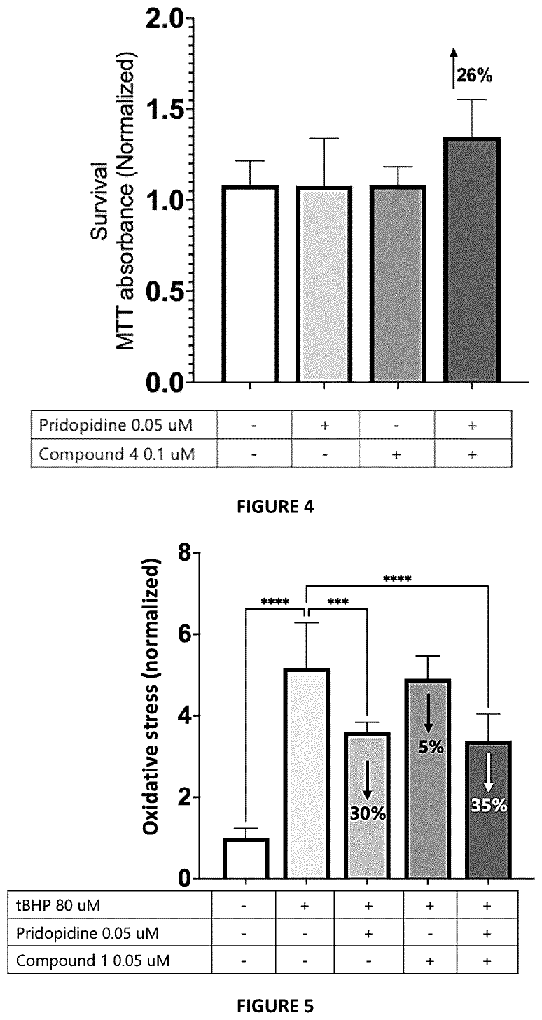

In ONHA following oxidative stress with tBHP (40 μM), neither pridopidine (0.05 μM) nor compound 4 (0.1 μM) have any effect on cell viability when administered individually. In combination, pridopidine and compound 4 show an unexpected and surprising synergistic effect, increasing cell viability by 26%. (FIG. 4)

### Pridopidine and Compound 1 Reduce Oxidative Stress in an Additive Manner

Incubation with tBHP (80 μM) increases oxidative stress 5-fold in ONHA. Pridopidine alone at 0.05 μM reduces oxidative stress by 30%. Compound 1 at the concentration of 0.05 μM reduces oxidative stress by 5%. In combination, pridopidine and compound 1 reduce oxidative stress by 35%, showing an enhanced, additive effect (FIG. 5).

In summary, the combination of pridopidine with compound 1 or compound 4 has a surprising and unexpected benefit on cell viability, showing increased viability that is far beyond the effect observed with each compound individually. The combination effect of pridopidine and Compound 1 can also be seen on oxidative stress, where the effect in combination is enhanced compared to each compound alone.

### CONCLUSIONS

The potential therapeutic effects of pridopidine were studied in two different experimental glaucoma models, in which retinal degeneration is induced by elevated IOP. Pridopidine demonstrated a neuroprotective effect on RGCs in both models. Surprisingly, pridopidine's protective effect was not mediated by lowering IOP, suggesting therapeutic potential for optic neuropathies beyond glaucoma.

The effects of pridopidine in combination with compounds 1 and 4, were further studied in neurons and in ocular tissues (ONHA). The combination of pridopidine and compound 1 or compound 4 showed a surprising and unexpected synergistic effect, both on BDNF secretion and on ONHA viability.

### REFERENCES

1) “Glaucoma”, Merck Manual of Diagnosis and Therapy (1999), Merck Research Laboratories, (Whitehouse Station, NJ), 733-738.
2) Alward, “Medical Management of Glaucoma”, N Eng J Med, 1998; 339:1298-1307).
3) Coleman “Glaucoma”, Lancet, 1999; 354:1803-1810.
4) CSID:25948790, www.chemspider.com/Chemical-Structure.25948790.html (accessed 23:27, Jul. 15, 2016).
5) CSID:7971505, www.chemspider.com/Chemical-Structure.7971505.html (accessed 23:33, Jul. 15, 2016).
6) Eddings, Chelsy R., Nicolas Arbez, Sergey Akimov, Michal Geva, Michael R. Hayden, and Christopher A. Ross. 2019. “Pridopidine Protects Neurons from Mutant-Huntingtin Toxicity via the Sigma-1 Receptor.” Neurobiology of Disease. https://doi.org/10.1016/j.nbd.2019.05.009.
7) Farkas et al., “Apoptosis, Neuroprotection and Retinal Ganglion Cell Death: An Overview”, Int Ophthalmol Clin 2001; 41:111-13 0.
8) Ionescu, Ariel, et al. 2019. “Targeting the Sigma-1 Receptor via Pridopidine Ameliorates Central Features of ALS Pathology in a SOD1G93A Model.” Cell Death & Disease 10 (3): 210. https://doi.org/10.1038/s41419-019-1451-2.
9) Kipnis et al., “T Cell Immunity To Copolymer 1 Confers Neuroprotection On The Damaged 5 Optic Nerve: Possible Therapy For Optic Neuropathies”, Proc Natl Acad Sci 2000; 97:7446-7451.
10) Mysona, Barbara, et al. 2017. “The Role of Sigma 1 Receptor as a Neuroprotective Target in Glaucoma.” Advances in Experimental Medicine and Biology. https://doi.org/10.1007/978-3-319-50174-1_20.
11) Ryskamp, Daniel A., Lili Wu, Jun Wu, Dabin Kim, Gerhard Rammes, Michal Geva, Michael Hayden, and Ilya Bezprozvanny. 2019. “Pridopidine Stabilizes Mushroom Spines in Mouse Models of Alzheimer's Disease by Acting on the Sigma-1 Receptor.” Neurobiology of Disease. https://doi.org/10.1016/j.nbd.2018.12.022.
12) U.S. Pat. No. 6,903,120, issued Jun. 7, 2005 (Sonesson, et al.)
13) U.S. Pat. No. 7,923,459, issued Apr. 12, 2011 (Gauthier, et al.)
14) PCT International Application Publication No. WO 2017/015609
15) Weinreb et al., “Is Neuroprotection a Viable Therapy for Glaucoma?” Arch Ophthalmol 1999; 117:1540-1544.
16) Maurya et al., Oxidative stress in retinal pigment epithelium degeneration: from pathogenesis to therapeutic targets in dry age-related macular degeneration. Neural Regeneration Research 18(10): p 2173-2181, October 2023.
17) Nadeem et al., Advancements in the treatment of geographic atrophy: focus on pegcetacoplan in age-related macular degeneration. Ann Med Surg (Lond). 2023 Nov. 1; 85(12):6067-6077.
18) Francardo, Veronica, Michal Geva, Francesco Bez, Quentin Denis, Lilach Steiner, Michael R Hayden, and M Angela Cenci. 2019. “Pridopidine Induces Functional Neurorestoration Via the Sigma-1 Receptor in a Mouse Model of Parkinson's Disease.” Neurotherapeutics, no. 16: 465-479.
19) Cheng, Yung-Chi, and William H. Prusoff. 1973. “Relationship between the Inhibition Constant (KI) and the Concentration of Inhibitor Which Causes 50 percent Inhibition (I50) of an Enzymatic Reaction.” Biochemical Pharmacology. https://doi.org/10.1016/0006-2952(73)90196-2.
20) Geva, Michal, Tal Birnberg, Brian Weiner, Andrew Lysaght, Yoonjeong Cha, Avia Merenlender-Wagner, Aric Orbach, et al. 2016. “Pridopidine Activates Neuroprotective Pathways Impaired in Huntington Disease.” Human Molecular Genetics 25 (18): 3975-87. https://doi.org/10.1093/hmg/ddw238.
21) Johnston, Tom H., Michal Geva, Lilach Steiner, Aric Orbach, Spyros Papapetropoulos, Juha-Matti Savola, Ian J. Reynolds, et al. 2019. “Pridopidine, a Clinic-Ready Compound, Reduces 3,4-Dihydroxyphenylalanine-Induced Dyskinesia in Parkinsonian Macaques.” Movement Disorders, December. https://doi.org/10.1002/mds.27565.
22) Katz, D M. 2014. “Brain-Derived Neurotrophic Factor and Rett Syndrome.” Handbook of Experimental Pharmacology 220: 481-95. https://doi.org/10.1007/978-3-642-45106-5_18.
23) Zuccato, Chiara, and Elena Cattaneo. 2009. “Brain-Derived Neurotrophic Factor in Neurodegenerative Diseases.” Nature Reviews Neurology 5 (6): 311-22. https://doi.org/10.1038/nrneurol.2009.54.

What is claimed is:

1. A method of treating, reducing or inhibiting a neurodegenerative eye disease or a symptom thereof in a subject comprising administering to the subject a composition comprising pridopidine or pharmaceutically acceptable salt thereof and at least one of compounds 1-8 or pharmaceutically acceptable salt thereof; wherein compounds 1-8 are represented by the following structures:

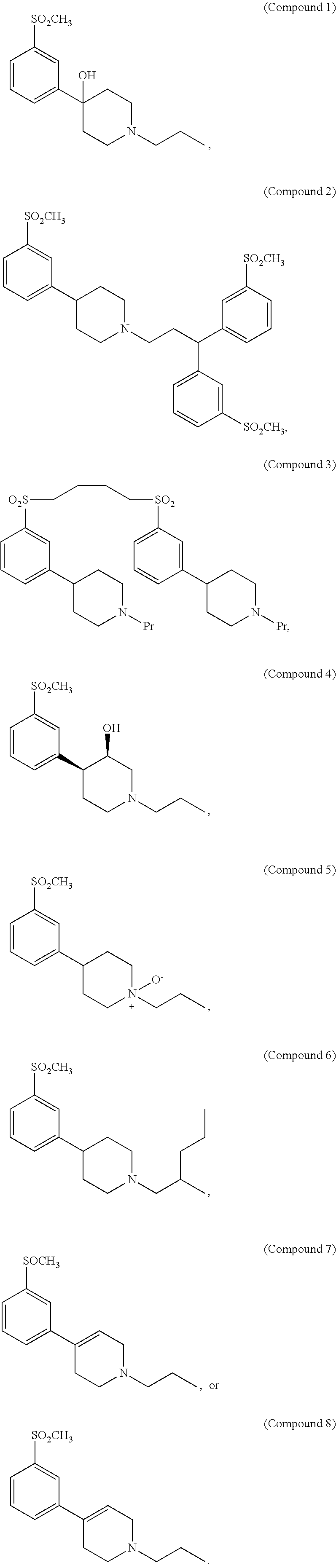

2. The method of claim 1, wherein the composition comprises pridopidine or pharmaceutically acceptable salt thereof and Compound 1 or pharmaceutically acceptable salt thereof.

3. The method of claim 1, wherein the composition comprises pridopidine or pharmaceutically acceptable salt thereof and Compound 4 or pharmaceutically acceptable salt thereof.

4. The method of claim 1, wherein the composition comprises pridopidine or pharmaceutically acceptable salt thereof and Compound 1 and Compound 4 or pharmaceutically acceptable salt thereof.

5. The method of claim 1, wherein the neurodegenerative eye disease is selected from the group consisting of glaucoma, Age-related Macular Degeneration (AMD), geographic atrophy (GA), optic neuropathy, Microphthalmia, syndromic 12 (MCOPS12) and retinitis pigmentosa.

6. The method of claim 5, wherein the Age-related Macular Degeneration is Wet Age-related Macular Degeneration (“Wet AMD”) or Dry Age-related Macular Degeneration (“Dry AMD”).

7. The method of claim 5, wherein the optic neuropathy is Leber hereditary optic neuropathy.

8. The method of claim 5, wherein the glaucoma is open-angle glaucoma, primary open-angle-glaucoma, angle-closure glaucoma, pigmentary glaucoma, pseudoexfoliative glaucoma, neovascular glaucoma, steroid-induced glaucoma, normal-tension glaucoma, pressure-independent glaucoma or any combination thereof.

9. The method of claim 1, wherein the symptom is retinal ganglion cell damage, retinal ganglion cell loss, an optic nerve axon loss or damage, macular degeneration, a retinal ganglion cell (RGC) loss or damage, a retinal pigment epithelium cell (RPE) loss or death or any combination thereof.

10. The method of claim 1, wherein the composition is effective in
reducing or preventing retinal ganglion cell (RGC) loss or damage in the subject,
improving retinal ganglion cell (RGC) viability in the subject,
reducing or preventing optic nerve axon loss or damage in the subject,
reducing or preventing optic nerve head astrocytes (ONHAs) loss or damage in the subject,
reducing oxidative stress in ONHAs,
improving optic nerve head astrocytes (ONHAs) viability in the subject,
increasing cell viability in the subject,
reducing or preventing retinal pigment epithelium cell (RPE) loss or death in a subject,
protecting an optic nerve axon from degeneration in the subject,
or any combination thereof.

11. The method of claim 10, wherein the axon degeneration is induced by elevated intraocular pressure.

12. The method of claim 10, wherein the cell viability is increased by at least 3%, by at least 5%, by at least 10%, by at least 20%, or by at least 25%.

13. The method of claim 10, wherein the optic nerve axon loss is reduced by at least 3%, by at least 5%, by at least 10%, by at least 20%, by at least 30%, by at least 40% or by at least 50%.

14. The method of claim 13, wherein the optic nerve axon loss is reduced by more than 50%, more than 60%, more than 70%, or more than 80%.

15. The method of claim 10, wherein the retinal ganglion cell loss is reduced by at least 10%, by at least 20%, by at least 30%, by at least 40% or by at least 50%.

16. The method of claim 15, wherein the retinal ganglion cell loss is reduced by more than 50%, more than 60%, more than 70%, or more than 80%.

17. The method of claim 10, wherein the optic nerve head astrocyte (ONHA) loss is reduced by at least 10%, by at least 20%, by at least 25%, by at least 30%, by at least 35%, by at least 40% or by at least 50%.

18. The method of claim 1, wherein the composition comprises a unit dose of between 22.5 mg to 315 mg.

19. The method of claim 18, wherein the composition is administered once a day, twice a day or three times a day.

20. The method of claim 1, wherein the composition is administered orally or topically.

21. The method of claim 1, wherein the composition is administered intraocularly, intravitreally, periocularly or ocularly.

22. The method of claim 21, wherein the composition is administered by an eye drop application to the conjunctiva.
